(19) United States
(12) Patent Application Publication
(10) Pub. No.: US 2010/0196311 A1
(43) Pub. Date: Aug. 5, 2010

(54) METHODS AND COMPOSITIONS CONTAINING mTOR INHIBITORS FOR ENHANCING IMMUNE RESPONSES

(76) Inventors: Hyung L. Kim, Los Angeles, CA (US); Protul Shrikant, Amherst, NY (US); YanPing Wang, Los Angeles, CA (US); Qingsheng Li, East Amherst, NY (US); Rajesh Rao, Amherst, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/687,650

(22) Filed: Jan. 14, 2010

Related U.S. Application Data
(60) Provisional application No. 61/144,537, filed on Jan. 14, 2009.
(60) Provisional application No. 61/293,096, filed on Jan. 7, 2010.

(51) Int. Cl. A61K 38/20; A61K 39/00; A61K 35/12; A61P 35/00

(52) U.S. Cl. 424/85.2; 424/277.1; 424/184.1; 424/93.71

(57) ABSTRACT

Provided are compositions and methods for enhancing immune responses to an antigen. The compositions contain an isolated population of CD8+T cells and an inhibitor of mammalian target of rapamycin (mTOR). The method for obtaining an enhanced immune response to an antigen in an individual entails administering to the individual the antigen and an inhibitor of mammalian target of rapamycin (mTOR). CD8+T cells may also be used for adoptive cell transfer (ACT) therapy.

This application claims priority to U.S. application No. 61/144,537 filed Jan. 14, 2009, and U.S. application No. 61/293,096, filed Jan. 7, 2010, the disclosures of each of which are incorporated herein in the entireties.

This invention was made with government support under grant no. 5R01CA104645 awarded by the National Institutes of Health. The government has certain rights in the invention.

### FIELD OF THE INVENTION

The present invention relates generally to modulating immune responses and more specifically to enhancing cell-mediated immune response in an individual using mammalian target of rapamycin (mTOR) inhibitors.

### BACKGROUND OF THE INVENTION

Cancer vaccines are being actively evaluated in clinical and preclinical studies. In principle, recruiting the immune system to target cancer is attractive. The immune system is capable of recognizing tumor-specific antigens and eradicating diseased cells while sparing normal tissue. However, the successful application of cancer vaccines to treat patients has remained elusive, and there is an ongoing and unmet need for improving the efficacy of cancer vaccines.

### SUMMARY OF THE INVENTION

The present invention provides compositions and methods for enhancing the efficacy of vaccines. In one embodiment, the invention provides a method for enhancing an immune response to an antigen in an individual. The method comprises administering to the individual the antigen and an mTOR inhibitor. The mTOR inhibitor and the antigen may or may not be administered as components of the same composition, and may be administered concurrently or sequentially. It is preferable to administer the mTOR inhibitor after administration of the antigen.

In another embodiment, the invention provides a composition comprising an isolated population of CD8+T cells and an inhibitor of mTOR. The composition may further comprise an antigen to which the CD8+T cells are specific, and may further comprise adjuvants, such as IL-12.

The enhanced immune response can comprise an enhanced cell mediated immune response against cells that bear the antigen in the individual. The enhanced response can include an increase in CD8+T cells that exhibit cytotoxic activity against cells that bear the antigen. The enhanced immune response may also or alternatively include CD8+T cells that exhibit enhanced sustenance and/or antigen-recall responses to the antigen, or an increase of the amount and/or activity of effector CD8⁺T cells that are specific for the antigen. Combinations of such immune responses may also be induced by the compositions and methods of the invention. The enhanced immune responses may manifest themselves as an inhibition of the growth of cells that express the antigen, death of antigen expressing cells in the individual, and/or by a prolongation of the survival of the individual, or any other way that will be known to those skilled in the art.

In various embodiments, the individual treated using the methods and compositions of the invention are individuals who are in need of an enhanced immune response to an antigen. The individual can be an individual who has not previously received an mTOR inhibitor. Non-limiting examples of such individuals include those who have underone immunosuppressive therapy for, for example, organ transplantations. In one embodiment, the individual is an individual in need of treatment for a cancer.

It is expected that the invention will be suitable for use with any mTOR inhibitor, and for enhancing a cell mediated immune response (which may or may not also comprise a humoral and/or innate immune response) against any antigen that can be presented to a CD8+T cell.

### BRIEF DESCRIPTION OF THE FIGURES

FIG. 1. Instructions that Program Naive CD8⁺T Cell for Type I Effector Maturation Enhances and Sustains mTOR Activity. (A and B) OT-I cells stimulated with BOK (Ag+B7.1) (±) IL-12 were evaluated for (A) IFN-γ by ICS and (B) cytolytic activity (primary, 72 hr poststimulation; secondary, 24 hr postsecondary stimulation); ***p<0.0002. (C-E) OT-I cells stimulated with antigen (Ag) (the antigen is a peptide consisting of Ser Ile Ile followed by Asn Phe Glu which are followed by Lys and Lue (OVAp), used at 10 nM) plus B7.1 (100 μg/ml)(Ag+B7.1)(±) IL-12 (2 ng/ml) were evaluated by ICS at the indicated time points for (C) phosphorylated mTOR, (D) phosphorylated S6K, and (E) phosphorylated ribosomal S6. For mTOR inhibition, rapamycin (20 ng/ml) was added 30 min prior to addition of antigen, cytokine Data are representative of at least three independent experiments with similar outcomes. (Data are presented as mean±SEM.)

FIG. 2. IL-12 Enhances Antigen-Induced mTOR Activity via PI3K and STAT4 (A and B) OT-I cells stimulated with Ag+B7.1 (±) IL-12 and LY294002 (10 μM) were evaluated by ICS for (A) phosphorylated Akt at 48 hr or (B) phosphorylated S6K at the indicated time points. (C) WT or Stat4⁻¹⁻ OT-I cells stimulated with Ag+B7.1 in the presence or absence of IL-12 were analyzed at the indicated time points for phosphorylated S6K; ***p<0.0001. Experiments shown are representative of three independent experiments with similar outcomes. (Data are presented as mean±SEM.)

FIG. 3. Sustained mTOR Activity Is Essential for Heritable Type I Effector Differentiation of CD8⁺ T Cells. (A-C) OT-I cells stimulated with Ag+B7.1 (±) IL-12 and rapamycin were evaluated at the primary and secondary phase for (A) IFN-γ by ICS; ′**p<0.0002; n.s., not significant; (B) cytolytic activity; or (e) granzyme B expression at 72 hr by ICS. (D and E) OT-I cells were stimulated with Ag+B7.1 (±) IL-12 and rapamycin was added 12 hr after stimulation to evaluate cells for (D) S6K phosphorylation at 48 hr and (E) IFN-γ production at the primary and secondary phase. Experiments shown are representative of at least three (A and B) and two (C-E) independent experiments with similar outcomes. (Data are presented as mean±SEM.)

FIG. 4. IL-12-Enhanced mTOR Phosphorylation Is Essential for T-bet-Determined Type I Effector Maturation of CD8⁺ T Cells. (A-C) OT-I cells stimulated with Ag+B7.1 (±) IL-12 and rapamycin were evaluated for (A) mRNA for T-bet at the indicated time points by RT-PCR, (B) T-bet protein expression at the indicated time-points by ICS, and (C) T-bet protein expression by ICS before and after antigen recall. **p<0.0035; ***p<0.0005. (D) WT and Tbx21⁻¹⁻ OT-I cells were stimulated with Ag+B7.1 (±) IL-12 and evaluated for IFN-γ production at the primary and secondary phase. ***p<0.0001. (E and F) OT-I cells stimulated with Ag+B7.1 (±) IL-12 and rapamycin were transduced with T-bet-ER retroviral vector (±) 4-HT (10 nM) and evaluated by ICS for (E) T-bet protein expression by ICS and (F) IFN-γ at secondary phase (168 hr). (G and H) OT-I cells stimulated with Ag+B7.1 (±) insulin (1 U/ml) and rapamycin were evaluated by ICS for (G) S6K phosphorylation at 48 hr and (H) T-bet expression at 72 hr. Experiments shown are representative of at least three (A, B, D, E, and F) and two (C, G, and H) independent experiments with similar outcomes. (Data are presented as mean±SEM).

FIG. 5. Inhibition of mTOR Promotes Persistent Eomes Expression and Phenotypic Markers of Memory in CD8+T Cells. (A and B) OT-I cells stimulated with Ag+B7.1 (±) IL-12 and rapamycin were evaluated for (A) mRNA for Eomes at the indicated time points by RT-PCR and (B) Eomes protein expression at 72 hr by ICS. *p<0.03. (C) OT-I cells stimulated with Ag+B7.1 (±) IL-12 and rapamycin were transduced with T-bet-ER retroviral vector (±) 4-HT (10 nM) and evaluated for Eomes protein expression at 96 hr. (D) OT-I cells stimulated with Ag+B7.1 (±) IL-12 and rapamycin were evaluated for CD62L, CD69, KLRG1, CD127, and CD122 expression at 72 hr. (E) Bcl-2 and Bcl-3 mRNA expression at the indicated time points. (F and G) OT-I cells stimulated with Ag+B7.1 (±) IL-12 and rapamycin for 72 hr were washed twice and rested for an additional 72 hr in the presence of (F) IL-7 (10 ng/ml), *p<0.02, and (G) IL-15 (10 ng/ml). *p<0.02 and percent (%) cell recovery was calculated at 144 hr. Experiments shown are representative of three independent experiments with similar outcomes. (Data are presented as mean±SEM.)

FIG. 6. Inhibition of mTOR Enhances Memory CD8⁺ T Cell Generation. OT-I cells (Thy1.1⁺) stimulated with Ag+B7.1 (±) IL-12 and rapamycin were harvested at 72 hr and adoptively transferred (2×10⁶ cells) into BL/6 recipients. (A) The absolute number of adoptively transferred OT-I cells in the lymph node, **p<0.0052; spleen, **p<0.0037; and liver, **p<0.0012 and **p<0.0011 at 24 hr post transfer. (B-E) The recipient mice were immunized with IFA-OVA on day 40 posttransfer and secondary CD8⁺ T cell responses were measured 3 days later (B) The absolute numbers of adoptively transferred cells before (day 40) and after (day 43) immunization in the spleen. The numbers in parenthesis indicate fold expansion of CD8α⁺Thy1.1⁺ from day 40 to day 43 and (C) absolute numbers of IFN-γ secreting CD8α*Thy1.1⁺ cells in the spleen on day 43; *p<0.01, **p<0.008. The numbers in parenthesis indicate the MFI of IFN-γ expression (D) and Granzyme B expression on CD8α⁺Thy1.1⁺ cells in the spleen on day 43 and (E) the in vivo antigen-specific cytolysis on day 43. A representative of two independent experiments is shown. (Data are presented as mean±SEM.).

FIG. 7. mTOR Inhibition Promotes CD8⁺ T Cell-Mediated Antitumor Immunity. (A and 8) Naive or 72 hr conditioned OT-I cells were adoptively transferred into B1/6 recipients. Mice were inoculated with 2×10⁶ E.G7 tumor cells 24 hr postadoptive transfer of OT-I cells. (A) Tumor size (mm³) over time from tumor inoculation and (8) percent of tumor-free survival over time from tumor inoculation is shown. A representative of two independent experiments is shown.

FIG. 8. Renal Cell Carcinoma model. mTOR inhibition with temsirolimus enhanced the antitumor effects of a cancer vaccine (complex of hsp110 and CA9) in Balb/C mice 10 days after implantation of RENCA tumors expressing the tumor antigen CA9. Each line represents tumor growth in a single mouse. To generate the vaccine, CA9 (antigen target) and HSP110 (heat shock protein adjuvant) were complexed at 1:1 molar ratio by incubating at 43° C. for 30 min. On day 0, 2×105 Renca-CA9 cells were implanted into mice. The vaccine was administered i.d. on days 10, 17 and 24. Temsirolimus was inject i.p. on days 11 to 16, 18 to 23 and 25 to 30.

FIG. 9. mTOR inhibition with temsirolimus enhanced the antitumor effects of a cancer vaccine (complex of gp100 and CA9) in C57/BL6 mice treated 10 days after implantation of B16 tumors expressing gp100. Each line represents tumor growth in a single mouse. To generate the vaccine, gp100 (antigen target) and CA9 (adjuvant) were complexed at 1:1 molar ratio by incubating at RT for 30 min. On day 0, 2×105 B16-gp100 cells were implanted into mice. The vaccine was administered i.d. on days 10, 17 and 24. Temsirolimus was inject i.p. on days 11 to 16, 18 to 23 and 25 to 30.

FIG. 10. Immunization with CA9+gp100 elicited a gp100-specific IFN-γ response measured using the ELISPOT assay.

FIG. 11 provides a graphical depiction of data showing that an mTOR inhibitor enhances immunization mediated protection against established ovarian tumors.

FIG. 12 provides a graphical depiction of data showing that an mTOR inhibitor enhances immunization mediated anti-thymoma efficacy.

FIG. 13 provides a graphical depiction of data showing that an mTOR inhibitor enhances homeostatic proliferation (HP)-induced anti-tumor immunity.

FIG. 14 provides a graphical depiction of data showing that an mTOR inhibitor enhances immunization mediated tumor protection.

FIG. 15 provides a graphical depiction of data showing that an mTOR inhibitor treatment enhances a HP-induced anti-tumor CD8+T cell response.

FIG. 16 provides a graphical depiction of data showing that an mTOR inhibitor enhances CD8+T cell mediated adoptive cell transfer (ACT) therapy of ovarian tumor.

### DESCRIPTION OF THE INVENTION

The present invention provides compositions and methods for modulating immune responses. In one embodiment, the invention provides a composition comprising an isolated population of CD8+T cells and an inhibitor of mammalian target of rapamycin (mTOR). The composition may further comprise an antigen to which the CD8+T cells are specific.

As used herein “CD8+” T cells means T cells that express CD8 (cluster of differentiation 8). CD8 is a well characterized transmembrane glycoprotein that serves as a co-receptor for T cell receptors (TCR). CD8 binds to the Class I major histocompatibility complex (MHC-I) protein on the surface of antigen presenting cells in humans.

In another embodiment, the invention provides a method for enhancing an immune response to an antigen in an individual comprising administering to the individual the antigen and an mTOR inhibitor. The antigen and the mTOR inhibitor are administered in an amount effective to enhance the immune response to the antigen in the individual. The mTOR inhibitor and the antigen may or may not be administered concurrently.

The enhanced immune response can comprise an enhanced cell mediated immune response against cells that bear the antigen in the individual. The enhancement can be relative to a control to whom the antigen, (and optionally any adjuvant), but not the mTOR inhibitor, has been administered. The enhanced cell mediated immune response can include but is not necessarily limited to an increase in CD8+T cells that exhibit cytotoxic activity against cells that bear the antigen, or CD8+T cells that exhibit enhanced sustenance and/or antigen-recall responses to the antigen, or an increase of the amount and/or activity of effector CD8⁺ T cells that are specific for the antigen, or combinations of the foregoing types of cell mediated immune responses. The enhanced cell mediated immune response elicited by the method of the invention may be accompanied by beneficial changes in humoral and/or innate immune responses. In one embodiment, an enhanced immune response can be evidenced by an inhibition of the growth of cells that express the antigen, death of antigen expressing cells in the individual, and/or by a prolongation of the survival of the individual.

In the present invention, we demonstrate that interleukin-12 (IL-12) enhanced and sustained antigen and costimulatory molecule (B7.1)-induced mTOR kinase activity in naive CD8⁺ (OT-I) T cells via phosphoinositide 3-kinase and STAT4 transcription factor pathways. However, blocking mTOR activity by a representative mTOR inhibitor (rapamycin) reversed IL-12-induced effector functions because of loss of persistent expression of the transcription factor T-bet. We show that rapamycin treatment of IL-12-conditioned OT-I cells promoted persistent Eomesodermin expression and produced memory cell precursors that exhibited enhanced sustenance and antigen-recall responses upon adoptive transfer. The memory cell precursors showed greater tumor efficacy than IL-12-conditioned effector OT-I cells. Thus, and without intending to be bound by any particular theory, it is considered that the present invention for the first time discloses that mTOR is the central regulator of transcriptional programs that determine effector and/or memory cell fates in CD8⁺ T cells.

In addition to discovering the role of mTOR in determining the developmental fate of CD8+T cells, we demonstrate that the addition of an mTOR inhibitor to a vaccine regimen can provide therapeutic and prophylactic benefits to an individual. In particular, we demonstrate in various embodiments of the invention using each of temsirolimus and rapamycin to enhance an immunological effect of cancer vaccines in animal models of cancer. In connection with this, temsirolimus is known to have direct antiproliferative (cytostatic) properties and is approved for treatment of advanced renal cell carcinoma (RCC). It has been suggested to use mTOR inhibitors with other cytostatic agents for treating cancer (T. Abraham and J. Gibbons; Clin Cancer Res (2007) 13:3109-3114), but the art does not teach or suggest using an mTOR inhibitor in combination with vaccines. To the contrary, temsirolimus and rapamycin are commonly used as immunosuppressive agents, and the art teaches that combining vaccines with agents having known immunosuppressive effects would be undesirable. For example, Spaner teaches against using rapamacyin as a cancer vaccine adjuvant because of its immunosuppressive properties, and indicates the same caveat could apply to other inhibitors of the mTOR related PI-3K pathway, which is believed to mediate cell-cycle progression of T cells. (Spaner, D. E., Journal of Leukocyte Biology Volume 76, August 2004, pp 338-351). Furthermore, among the T cell types responsible for peripheral tolerance and immune suppression, regulatory T cells (Tregs) are believed to be critical. Naturally occurring regulatory T cells represent 5-10% of total CD4+T cells and can be defined based on expression of CD25 and FOXP3 (Sakaguchi S: Nat Immunol 6:345-52, 2005). However, the art indicates that inhibition of mTOR function results in expansion of murine Tregs both in vitro and in vivo. (Battaglia M, et al. Blood 105:4743-8, 2005; Battaglia et al: Diabetes 55:1571-80, 2006). Moreover, in humans, mTOR inhibition has been shown to promote expansion of Tregs in vitro and to enhance the suppressive capacity of Tregs in vivo (Monti P, et al. Diabetes 57:2341-7, 2008). Thus, the expectation from the state of the art is that combining an mTOR inhibitor with a vaccine would be detrimental to generating an immune response to the antigenic component of the vaccine, and therefore would not be expected to enhance or otherwise augment the immune response to the antigenic component of the vaccine. However, we unexpectedly discovered that the addition of mTOR inhibitors to cancer vaccine regimens improves the immunological response against antigenic components of the vaccine, inhibits cancer cell growth via immune mediated responses, and can prolong survival relative to controls. Thus, given the well-established role for mTOR inhibitors as immunosuppressants, the present discovery that mTOR inhibition enhances immune responses against cancer antigens when combined with vaccination regimens was surprising. In this regard, we demonstrate in particular that mTOR inhibitors (rapamycin and temsirolimus) can enhance the efficacy of cancer vaccines in established murine models for RCC and melanoma. Further, also using established murine models, we demonstrate that rapamycin enhances immunization mediated protection against ovarian tumors and thymoma. Further still, we demonstrate that rapamycin treatment can enhance homeostatic proliferation (HP) induced anti-tumor immunity, and can also provide a prophylactic benefit against tumor challenges based on induction of durable immunological memory. Thus, the present invention provides a heretofore unavailable and surprisingly effective method for enhancing the efficacy of vaccines, and in particular, cancer vaccines. Thus, it is expected that the present invention can enhance any vaccination regimen that operates at least in part through a cell mediated immune response.

In one embodiment, the method of the invention is performed for an individual who is in need of an enhanced immune response to an antigen.

In one embodiment, the individual is an individual who has not undergone immunosuppression therapy with an mTOR inhibitor. Non-limiting examples of such individuals include those who have not been treated for autoimmune disorders or organ transplantations using mTOR inhibitors. The individual may be one who is suspected of having a cancer, has been diagnosed with a cancer, or is at risk of developing a cancer based upon, for example, a genetic predisposition or behavioral or occupational risk factors.

mTOR is a well characterized protein which in humans is encoded by the FRAP1 gene. Its nucleotide coding and amino acid sequences are known in the art and can be accessed via GenBank accession no. BC117166, Jun. 26, 2006 entry, which is incorporated herein by reference.

It is expected based upon the disclosure presented herein that any mTOR inhibitor will be suitable for use in the compositions and methods of the invention, and that any mTOR protein expressed by any individual will be a suitable target for the inhibitors. It is preferable to use inhibitors that have selectivity and/or specificity for inhibition of mTOR, as opposed to broad spectrum kinase inhibitors. Thus, in various non-limiting embodiments, the mTOR inhibitor may be rapamycin, temsirolimus, everolimus torin and deforolimus, analogs of the foregoing, and combinations of the mTOR inhibitors and/or analogs thereof.

It is also expected that any antigen is suitable for use in the present invention, so long as the antigen comprises an amino acid sequence suitable for presentation by antigen presenting cells in conjunction with MHC class I molecules. Thus, the antigen may be or may comprise a protein or a peptide. The antigen may be a recombinant antigen, it may be chemically synthesized, it may be isolated from a cell culture, or it may be isolated from a biological sample obtained from an individual. The antigen may be present on cells in an infectious organisms or the antigen may be expressed by a diseased or infected cell, tissue or organ. The desired antigen may be well characterized, but may also be unknown, other than by its known or predicted presence in, for example, a lysate from a particular cell or tissue type. Antigens useful for the invention may be commercially available or prepared by standard methods.

In one embodiment, the antigen is a tumor antigen. Tumor antigens can be commercially available antigens, or they can be obtained by conventional techniques, such as by recombinant methods, or by preparation of tumor cell lysates. Antigens from the tumor lysates may be isolated, or the lysates themselves may be used as the antigen(s). The antigen can be used in a purified form or in partially purified or unpurified form. “Purified” as used herein means separated from other compounds or entities. The antigen may be added to a composition of the invention and/or used in the method of the invention as an unpurified, partially purified, substantially purified, or pure antigen. The antigen is considered purified when it is removed from substantially all other compounds, i.e., is pat least about 90%, 91%, 92%, 93%, 94%, 95%, 96%, 97%, 98%, 99%, or greater than 99% pure. A partially or substantially purified antigen may be removed from at least 50%, at least 60%, at least 70%, or at least 80% or more of the material with which it is naturally found, e.g., cellular material such as other cellular proteins, membranes, and/or nucleic acids.

In various embodiments, the cancer cell antigen may be expressed by cancer cells, specific examples of which include but are not limited to fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, and heavy chain disease.

The antigen may be an antigen that is expressed by an infectious agent or infectious organism, non-limiting examples of which include viruses, bacteria, fungi, protozoans, or any other parasite or otherwise infectious agent.

In another embodiment, the invention provides a method for enhancing in an individual an immune response to a desired antigen comprising administering to the individual a composition comprising CD8+T cells specific for the antigen and an effective amount of an inhibitor of mTOR.

The isolated CD8+T cells may be specific for but naïve with respect to the antigen, or they may have encountered the antigen to which an enhanced immune response is desired prior to being used in the method of the invention. Alternatively, the isolated CD8+T cells may be exposed to the desired antigen prior to administering them to the individual, such as by incubating the CD8+T cells with antigen presenting cells that present the antigen to the CD8+T cells. The CD8+T cells may be isolated from the individual in whom an enhanced immune response to a desired antigen is intended using any of a wide variety of well known techniques and reagents. Accordingly, the CD8+T cells can be re-introduced into the individual for performing the method of the invention.

In another embodiment, the invention provides compositions comprising an isolated population of CD8+T cells and an mTOR inhibitor. The CD8+T cells are specific for the antigen against which an enhanced immune response is desired. The composition is suitable for use in the method of the invention, since exposure of the CD8+T cells to the mTOR inhibitor imparts to them the capability to participate in an enhanced cell mediated immune response against the antigen when the CD8+T cells are introduced back into the individual and encounter the antigen. The isolated CD8+T cells may constitute various percentages of the cells in the composition. For example, the CD8+T cells may constitute at least 1%, 10%, 20%, 30%, 40%, 50%, 60%, 70%, 80%, 90%, 95%, or 100%, including all integers there between, of the T cells or total cells in the composition

The composition comprising the CD8+T cells and the mTOR inhibitor may further comprise the antigen. When the antigen is present in the composition comprising the isolated CD8+T cells, the antigen may be present as an independent entity, or in any context by which the antigen can interact with the T cell receptor (TCR) present on the CD8+T cells. When the antigen can interact with the TCR of the CD8+T cells the CD8+T cells can become activated. Examples of various embodiments by which the antigen can be provided in the composition such that it can be recognized by the CD8+TCR include but are not limited to it the antigen being present in association with MHC-I (or the equivalent presentation in an animal model) on the surface of antigen presenting cells, such as dendritic cells. Alternatively, the antigen could be in physical association with any other natural or synthesized molecule or other compound, complex, entity, substrate, etc., that would facilitate the recognition of the antigen by the TCR on the CD8+T cells. For example, the antigen could be complexed to a MHC-I or other suitable molecule for presenting the antigen to the CD8+TCR, and the MHC-I or other suitable molecule (e.g., K^{b} in the case of a composition comprising C57BL/6 murine CD8+T cells) could be in physical association with a substrate, such as a latex bead, plastic surface of any plate, or any other suitable substrate, to facilitate appropriate access of the antigen to the CD8+T cell TCR such that the antigen is recognized by the CD8+T cell. The composition may further comprise any of a variety of well know co-stimulatory molecules. It will be recognized by those skilled in the art that the compositions described herein are suitable for preparing the CD8+T cells for administration to an individual and/or could be administered directly to an individual, or could be further purified, combined, treated or mixed with any other of a variety of agents and/or processes that would render the compositions suitable for administration to an individual for the purposes of providing a therapeutic or prophylactic enhancement of a vaccine regimen against any desired antigen against which a cell mediated immune response could arise. In one embodiment, the composition also comprises cytokines, such as IL-12.

Methods for obtaining biological samples and isolating CD8+T cells from the samples are well known in the art. For example, routine cell sorting techniques that discriminate and segregate T cells based on T cell surface markers can be used to obtain an isolated population CD8+T cells for including in the compositions and methods of the invention. For example, a biological sample comprising blood and/or peripheral blood lymphocytes can be obtained from an individual and CD8+T cells isolated from the sample using commercially available devices and reagents, thereby obtaining an isolated population of CD8+T cells. The CD8+T cells may be further characterized and/or isolated on a phenotypic basis via the use of additional cell surface markers., such as CD44, L-selectin (CD62L), CD122, CD154, CD27, CD69, KLRG1, CXCR3, CCR7, IL-7Ra. The cells may also be initially isolated by negatively selecting CD4+/NK1.1+, B220, CD11b+, CD19+cells. The cells maybe naïve (CD62Ll hi, CD44 low, IL-7Ra hi, CD122 low, or antigen experienced; CD62L (low-moderate), CD44 hi, IL-7Ra (high or low) and CD122 moderately hi The isolated population of CD8+T cells can be mixed with the mTOR inhibitor and/or antigen in any suitable container, device, cell culture media, system, etc., and can be cultured in vitro and/or exposed to the one or more antigens, and any other reagent, or cell culture media, in order to expand and/or mature and/or differentiate the T cells to have any of various desired characteristics, such characteristics being known to those skilled in the art. For example, the isolated CD8+T cells may be treated so as to develop cytotoxic activity towards cells that bear an antigen to which an enhanced immune response would be desirable, the CD8+T cells could have enhanced sustenance and/or antigen-recall responses to presentation of the antigen, or the CD8⁺ T cells could have functional and/or phenotypic characteristics of effector T cells.

Compositions of the invention may comprise pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins. The compositions may further comprise any suitable adjuvant., including but not limited to immunological adjuvants that stimulate Toll-like (TLR), NLR and all DAMPS and PAMPS including incomplete freund's adjuvant, complete freund's adjuvant, Salmonella flagellin peptide/protein, CpG containing DNA, uric acid crystals, emulsion oils, viral vectors, RNA, and/or ssDNA, which can be used to add mix with antigen or inject into antigen provided hosts.

Those skilled in the art will recognize how to formulate dosing regimens for performing the method of the invention, taking into account such factors as the molecular makeup of the antigen, the size and age of the individual to be treated, and the type and stage of a disease with which the individual may be suspected of having or may have been diagnosed with.

The antigen and the mTOR inhibitor can be administered concurrently as components of the same composition. It is preferable to administer the mTOR inhibitor after administering the antigen to the individual. For example, the initial mTOR inhibitor administration can occur from several hours after administration of the antigen, and up to 60 days post antigen administration, including all days and hours there between. Further, it is preferable to provide repeated administrations of the mTOR inhibitor. For example, in one embodiment of the method of the invention, the mTOR inhibitor is administered at least once daily, and for a period of at least one weak. The mTOR inhibitor may be administered daily for longer than one week, for example, from 8-60 days, including all integers there between. In one embodiment, the mTOR inhibitor is administered for not more than 20 days, since we have determined that administration for more than 20 days reduces the enhancement effect.

The amount of mTOR inhibitor to be included in a composition of the invention and/or to be used in the method of the invention can be determined by those skilled in the art, given the benefit of the present disclosure. In certain embodiments, a composition comprising 15 μg of rapamycin administered once daily for 5-8 days is effective to enhance an immune system mediated effect in mouse models of cancers. It is expected that this amount of mTOR inhibitor and dosing regimen can be scaled accordingly for any given human patient and any given mTOR inhibitor based upon, for example, a mg/kg of bodyweight basis.

The method of the invention can be performed in conjunction with conventional therapies that are intended to treat a disease or disorder associated with the antigen. For example, if the method is used to enhance an immune response to a tumor antigen in an individual, treatment modalities including but not limited to chemotherapies, surgical interventions, and radiation therapy can be performed prior to, concurrently, or subsequent to the method of the invention.

The following Examples are intended to illustrate but not limit the invention.

### Example 1

This Example provides a description of the materials and methods used to obtain the data in Examples 2-9.

Mice and Reagents The C57BL/6, CD4⁺ TCR transgenic Rag2⁻¹⁻ (OT-II), CD8⁺ TCR transgenic Rag2⁻¹⁻ (OT-I, WT), Stat4⁻¹⁻ OT-I Rag2⁻¹⁻, and Tbx21⁻¹⁻ OT-I Rag2⁻¹⁻ mice were bred, housed, and used according to IACUC guidelines at RPCI. The rmIL-12 (2 ng/ml) was a gift from Wyeth, Inc. (Cambridge, Mass.). IFN-α was a gift from T. Tomasi (RPCI). rmIL-7 was purchased from Peprotech (Rocky Hill, N.J.). 2-DG, 4-HT, and rapamycin were purchased from Sigma Aldrich (St. Louis, Mo.). LY290042 was purchased from Calbiochem. Insulin was purchased from Novo Nordisk Inc. (Princeton, N.J.).

Stimulation of OT-1 Cells. Naive OT-I cells were stimulated with latex microspheres expressing H-2K^{b}/ovalbumin antigen and B7.1 according to known techniques. Naive OT-II cells were stimulated with anti-CD3-/anti-CD28-coated latex beads. In some experiments, the cell line derived from embryonic fibroblasts, namely, BOK (MEC.B7.SigOVA: expressing H-2K^{b}, OVAp and B7.1, were used as antigen-presenting cells to stimulate naive OT-I cells according to known techniques.

Evaluation of Secondary Antigen-Recall Responses In Vitro. For studying secondary antigen-recall response in vitro; OT-I cells harvested at 72 hr (primary) were washed thrice with medium and recultured (1×10⁵/ml) for an additional 72 hr in a 24-well plate with IL-7 (10 ng/ml) only. At 144 hr the cells were harvested, washed thrice with medium, had numbers adjusted (5×10⁵), and were restimulated with Ag/B7.1 only for an additional 24 hr (secondary stimulation). At 168 hr, the cells recovered were evaluated by flow cytometry and in vitro functional assay.

Retroviral Transduction and T-Bet Induction. T-bet-ER RV (Estrogen Responsive Retro Viral Vector) was cotransfected into Platinum-E cells together with the retroviral packaging vector pCL-Eco using LipoD293 DNA in vitro transfection reagent (SignaGen Laboratories) following the manufacturer's instructions. The medium was replaced the following day, and retroviral supernatant was collected 3 days after transfection. For transduction, naive OT-I cells stimulated for 24 hr, were suspended in retroviral supernatant containing polybrene (8 μg/ml; Sigma-Aldrich), and were spin-transduced at 2200 rpm for 90 min at 30° C. After spin-transduction, cells were cultured in fresh medium containing the same polarizing milieu as before, along with the addition of 4-HT (10 nM). At the end of 72 hr after initial stimulation, cells were washed thrice and maintained in the absence of any stimulation but in the presence of 4-HT and IL-7 (10 ng/ml).

Statistical Analysis. For statistical analysis, the unpaired Student's t test was applied. Tumor survival between various groups was compared using Kaplan Meier survival curves and log-rank statistics. Significance was set at p<0.05.

### Example 2

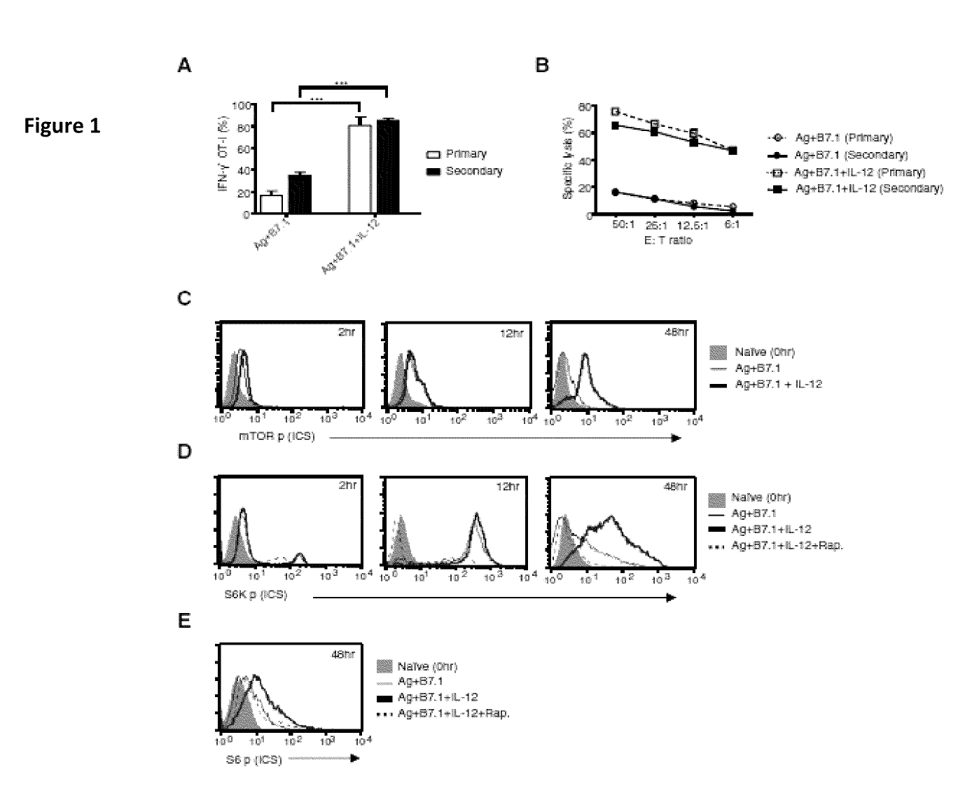

This Example demonstrates that instructions that program naive CD8⁺ T cells for Type I effector differentiation enhance mTOR activity. To characterize mechanisms underpinning instructional (signals 1, 2, and 3-antigen [Ag], B7.1 [costimulation], and IL-12 [cytokine], respectively) programming of naive CD8⁺ T cells for type I effector functions, we initiated our studies to confirm the deterministic role of IL-12 in imparting type I effector maturation in OT-I cells stimulated with adherent cell line, namely BOK expressing H-2K″, OVAp, and B7.1. Addition of IL-12 resulted in robust IFN-γ production and cytotoxic T lymphocyte (CTL) activity in OT-I cells at 72 hr (FIGS. 1 A and 1 B; primary). Furthermore, when the primary effector OT-I cells (72 hr) were rested with IL-7 for an additional 72 hr (12% IFN-γ detected at 144 hr) and restimulated with Ag and B7.1 (see Example 1), only the IL-12-conditioned OT-I cells reinduced IFN-γ and CTL activity (FIGS. 1A and 1B; secondary). Thus, IL-12 has a deterministic role in CD8+ T cell effector maturation.

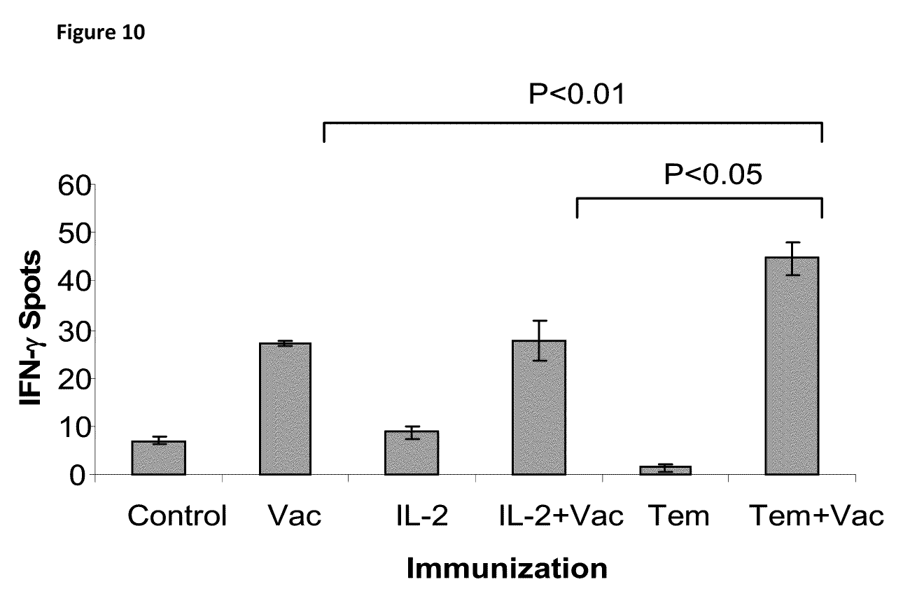

Although the kinase mTOR has been implicated as an integrator of various extracellular signals and a sensor for internal energy levels for determination of cell fate, the role for mTOR in integrating instructions that program naive CD8⁺ T cells for type I effector differentiation is unclear. First, we tested the ability of Ag and B7.1 (Ag+B7.1) in the presence or absence of IL-12 to activate mTOR in OT-I cells at various time points after stimulation. Stimulation of naive OT-I cells with Ag+B7.1 induced mTOR phosphorylation (activation) by 2 hr, which was maximal at 12 hr and barely detectable by 48 hr (FIG. 1C). Remarkably, IL-12 addition enhanced Ag+B7.1-induced mTOR phosphorylation at 2 hr, which was maintained at 48 hr after stimulation (FIG. 1C). Thus, although Ag+B7.1 induces mTOR phosphorylation, the addition of IL-12 enhances and sustains mTOR phosphorylation in OT-I cells. To verify that the induction of mTOR phosphorylation also led to its kinase activity, we monitored the kinetics of p70 S6K phosphorylation (Ser 371), a direct target of mTOR kinase activity. Although both Ag+B7.1 and Ag+B7.1 plus IL-12 induced similar amounts of S6K phosphorylation at 12 hr (maximal), the presence of IL-12 was able to sustain the S6K phosphorylation up to 48 hr (FIG. 10), in correlation with mTOR phosphorylation (FIG. 1C). Similarly, phosphorylation of S6 (Ser235 and -236), a downstream substrate of S6K, was also enhanced and sustained in IL-12-conditioned OT-I cells (FIG. 1E). The Ag+B7.1±IL-12 stimulation-induced S6K and S6 phosphorylation in OT-I cells was blocked by rapamycin (specific inhibitor of mTOR complex-1) (FIGS. 1D and 1E), thus confirming the ability of instructions to activate mTOR and its kinase activity in OT-I cells. The induction of blast transformation and CD98 expression in Ag+B7.1-stimulated OT-I cells was further augmented by IL-12 in a rapamycin sensitive manner. These observations identify mTOR as a target of instructions that program CD8⁺T cell effector responses and suggest a potential role for mTOR kinase in regulating IL-12-determined type I differentiation of CD8⁺T cells.

### Example 3

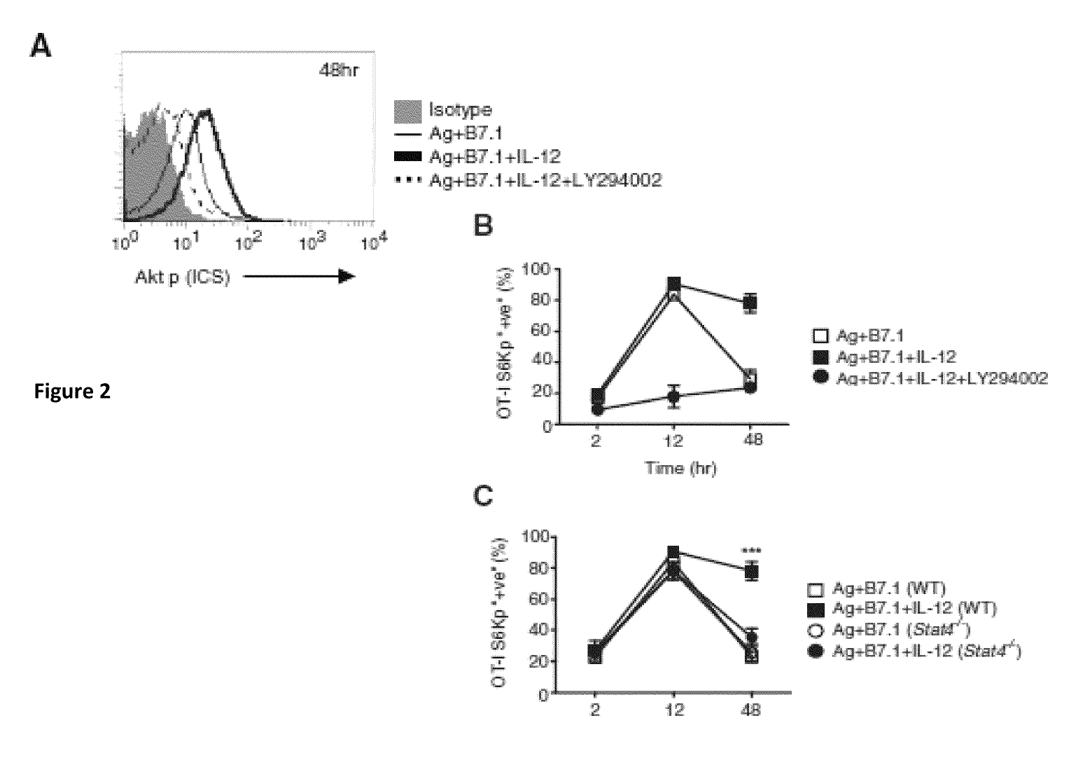

This Example demonstrates 11-12-enhanced mTOR activity in CD8⁺T cells requires PI3K and STAT4. To determine the molecular pathways governing mTOR activity in CD8⁺T cells, we analyzed whether the Ag-, B7.1-, and IL-12-induced phosphoinositide 3-kinase (PI3K)-Akt kinase pathway is required for mTOR signaling in CD8⁺T cells. The OT-I cells stimulated with Ag+B7.1±IL-12 were evaluated for Akt phosphorylation (Thr 308) as a functional measure of PI3K activity. Although Ag+B7.1 stimulation in the presence or absence of IL-12 induced similar amounts of Akt phosphorylation by 30 min, the presence of IL-12 augmented Akt phosphorylation up to 48 hr, which was blocked by the PI3K inhibitor (LY294002) (FIG. 2A), thereby confirming that IL-12 augments Ag+B7.1-induced PI3K activity in OT-I cells. Moreover, IL-12 augmented mTOR activity (S6K phosphorylation observed at 2, 12, and 48 hr) was blocked by PI3K inhibition (FIG. 2B), demonstrating that Ag+B7.1 and IL-12-activated PI3K activity in antigen-stimulated OT-I cells is required for induction of mTOR kinase activity.

The ability of IL-12 to instruct CD8+T cells for robust effector maturation requires STAT4 transcription factor. To determine whether IL-12 augmented mTOR activity in OT-I cells is STAT4 dependent, we tested the ability of wild-type (WT) or Stat4⁻¹⁻ OT-I cells to induce S6K phosphorylation upon stimulation with Ag+B7.1±IL-12. In contrast to our observations with PI3K inhibition, the absence of STAT4 in OT-I cells did not affect IL-12-induced S6K phosphorylation at early time points (2 hr and 12 hr) but failed to maintain the induced amounts of S6K phosphorylation (48 hr) (FIG. 2C). Thus, IL-12-induced PI3K and STAT4 have different roles in regulating mTOR activity in OT-I cells.

### Example 4

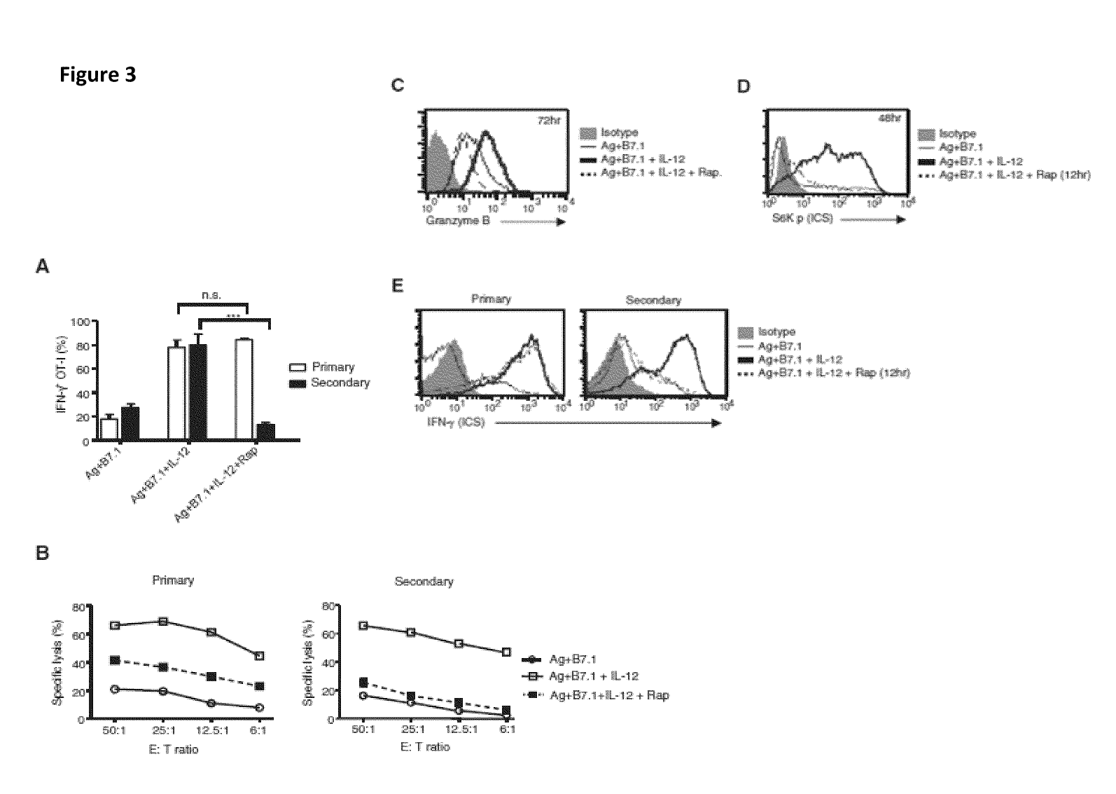

This Example demonstrates that sustained mTOR activity is essential for heritable Type I effector functions. Because the presence of IL-12 during antigen stimulation augments mTOR activity and is deterministic for type I effector maturation, we analyzed whether sustained mTOR kinase activity is required for IL-12-programmed type I effector functions in OT-I cells. To do so, we stimulated naive OT-I cells with BOK±IL-12, and rapamycin and effector functions were analyzed from the primary and secondary activated OT-I pool. Addition of rapamycin to IL-12-conditioned OT-I cells did not affect IFN-γ production from primary activated OT-I cells but reduced their CTL activity associated with decreased Granzyme B expression (FIGS. 3A, 3B, and 3C; primary). In contrast, we noted a complete reversal of IL-12-conditioned effector functions from the secondary activated pool (IFN-γ production and CTL activity) (FIGS. 3A and 3B; secondary). This blockade of IL-12-conditioned type I effector functions was not because of rapamycin-induced inhibition of cell proliferation and/or protein synthesis because reactivation of these cells in the presence of IL-12 resulted in considerable IFN-γ production. These results indicate that IL-12-induced commitment of naive CD8⁺ T cells for type I effector functions requires mTOR activity. In addition, we observed a block in IL-12-induced IFN-γ production and CTL activity upon rapamycin treatment at 144 hr after primary stimulation. These results further confirm that rapamycin treatment blocks type I effector functions, and the loss of effector functions observed in the secondary activated pool (168 hr) (FIGS. 3A and 3B; secondary) is believed to be solely because of the inability of these cells to reinduce IFN-γ production and not because of a refractory state of the rapamycin-treated cells.

To determine whether sustained mTOR activity achieved by IL-12 treatment was required for type I effector functions, we blocked persistence of IL-12 induced mTOR activity by adding rapamycin at 12 hr (mTOR activation peaks at 12 hr; FIGS. 1C and 1D) (FIG. 3D) after Ag+B7.1 stimulation and evaluated their ability to produce IFN-γ production from the primary and secondary activated OT-I pool. The addition of rapamycin at 12 hr blocked IL-12-induced effector functions, just as observed with the treatment at 0 hr (FIG. 3E; primary activated versus secondary activated responses). Thus, mTOR activity induced during the first 12 hr may not be sufficient to program CD8⁺ T cells for type I effector function and indicates importance of IL-12 induced persistence of mTOR activity (12 hr or later) to program type I effector functions in CD8⁺ T cells.

### Example 5

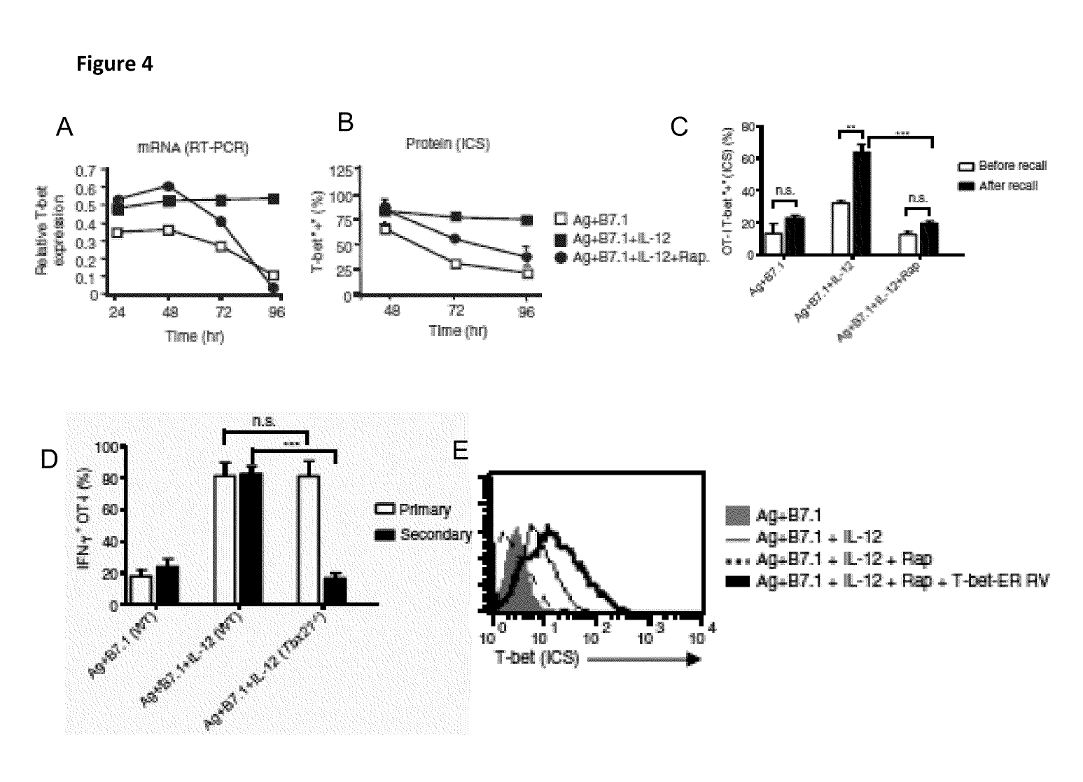

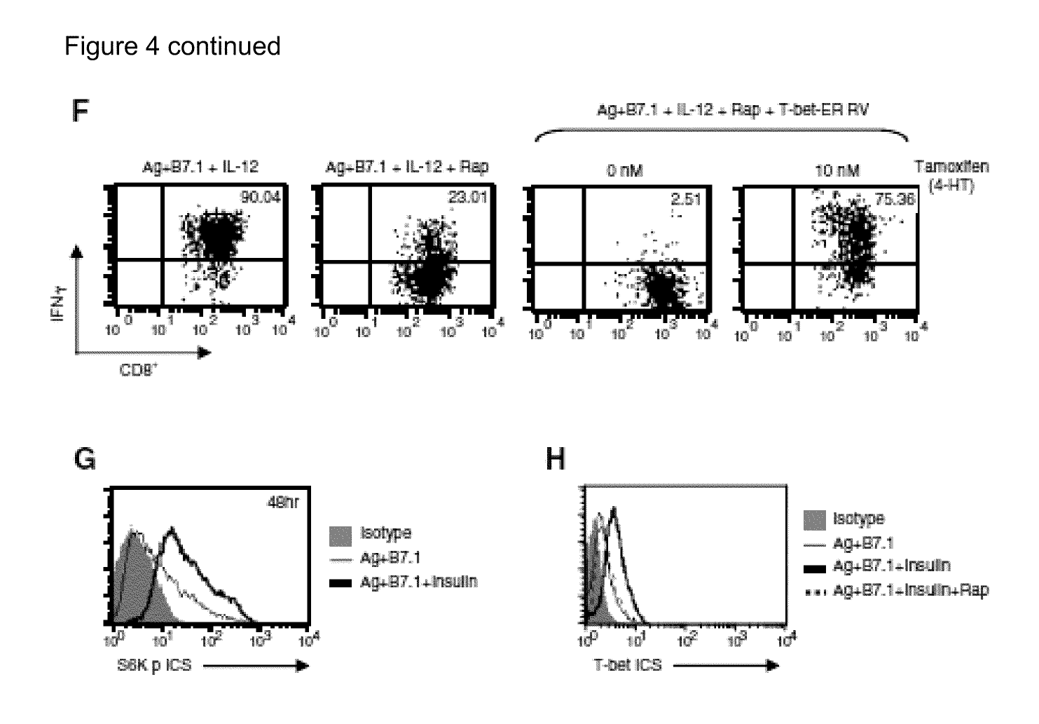

This Example demonstrates that IL-12 augmented mTOR activity is important for sustained T-bet expression. Because the sustained expression of T-bet is necessary and sufficient for imprinting type I effector cell fate (Matsuda et al., 2007) and mTOR inhibition reversed IL-12 imprinted type I effector maturation in OT-I cells (FIG. 3), we next sought to determine whether rapamycin treatment affects T-bet expression in OT-I cells by performing kinetic analysis of T-bet mRNA expression (FIG. 4A). The addition of IL-12 enhanced and sustained Ag+B7.1-induced T-bet expression at all time points tested (24-96 hr). However, mTOR inhibition did not affect Ag+B7.1 plus IL-12-induced early T-bet expression (24-48 h) but blocked IL-12-induced sustained T-bet mRNA expression (barely detectable by 96 hr), and correspondingly, the OT-I cells lost T-bet protein expression (FIG. 4B). Moreover, inhibition of mTOR activity at 12 hr was also able to achieve loss in T-bet expression, similar to the observed loss in type I effector maturation (FIG. 3E). Thus, IL-12 augmented (enhanced and sustained) mTOR activity is required for sustained T-bet expression in CD8⁺ T cells.

To demonstrate that rapamycin-mediated blockade of IFN-γ production during antigen recall was because of its inability to reinduce T-bet expression, we rendered IL-12-conditioned OT-I cells (72 hr type I effector cells) quiescent by IL-7 treatment for 72 h4 (144 hr) and evaluated their T-bet expression before (144 hr) and after (168 hr) antigen recall. Moderate T-bet expression was detected in OT-I cells conditioned primarily with Ag+B7.1 plus IL-12 at 144 hr, which was blocked upon rapamycin treatment (FIG. 4C). Notably, upon antigen recall, the IL-12-conditioned OT-I cells reinduced significantly higher amounts of T-bet protein, which was sensitive to rapamycin treatment (FIG. 4C). These observations demonstrate that rapamycin treatment blocks persistent T-bet expression, which may result in the block of IL-12-mediated type I effector maturation. This conclusion was supported by the fact that IL-12 conditioning of Tbx21⁻¹⁻ OT-I cells also failed to generate type I effector functions (FIG. 4D, secondary), although their ability to produce IFN-γ in the primary phase was not affected (FIG. 4D, primary). These observations are in agreement with rapamycin-treated IL-12-conditioned OT-I cells (FIG. 3A) and lend further support to our argument that the loss of persistent T-bet expression upon mTOR inhibition blocks IL-12-conditioned type I effector differentiation in CD8⁻¹⁻ T cells.

To directly determine whether the loss of T-bet expression upon rapamycin treatment led to loss of type I effector functions, we induced ectopic expression of T-bet in rapamycin-treated IL-12-conditioned OT-I cells and evaluated their ability to reinduce IFN-γ production from the secondary activated OT-I pool. The retroviral vector, T-bet-ER (T-bet-ER RV), was employed wherein the expression of T-bet is regulated by tamoxifen (4-HT) (Matsuda et al., 2007). Indeed, addition of tamoxifen (Tm, 10 nM) to T-bet-ER-transduced OT-I cells led to a substantial increase in T-bet expression (FIG. 4E) and restored IFN-γ production in rapamycin-treated IL-12-conditioned OT-I cells (FIG. 4F). Thus, demonstrating that IL-12-induced persistent mTOR phosphorylation is essential for sustained T-bet expression and T-bet-dependent type I effector commitment of CD8⁻¹⁻ Tcells.

The metabolic hormone insulin acts via insulin receptor substrate (IRS) to activate mTOR kinase, whereas 2-deoxyglucose (2-DG), a glycolytic inhibitor, leads to a blockade of mTOR activity. Therefore, we employed insulin and 2-DG to metabolically regulate mTOR activity and test whether they could impact T-bet expression in OT-I cells. Indeed, insulin addition to Ag+B7.1-stimulated OT-I cells enhanced mTOR activity (S6 Kp) and mTOR-dependent increase in T-bet expression (FIGS. 4G and 4H), whereas 2-DG addition to Ag+B7.1 and IL-12-stimulated OT-I cells led to loss of mTOR activity and T-bet expression. These results identify mTOR as a critical integrator of instructions to regulate T-bet expression in CD8⁺T cells.

### Example 6

This Example demonstrates differential requirements of mTOR kinase in CD4⁺ and CD8⁺Cells. Because treatment of CD4⁺T cells with rapamycin induces anergy and/or deviation to the Foxp³-expressing T regulatory cells, we analyzed whether inhibition of Ag+B7.1 and IL-12-induced mTOR activity interferes with CD8⁺T cell type I effector differentiation, because of block in activation, proliferation, and/or causes deviation to different effector subtypes. In agreement with published observations in CD4⁺T cells, our results demonstrate that rapamycin treatment significantly reduced activation (CD44 expression), proliferation (CFSE dilution), and cell recovery of CD4⁺T cells (OT-II). However, rapamycin treatment did not affect CD8⁺T cell (OT-I) early (CD69, 12 hr) and late activation (CD44) and only marginally affected proliferation (CFSE) and cell recovery. Moreover, in contrast to the reported expression of FoxP3 in CD4⁺T cells, the rapamycin-treated OT-I cells failed to persistently express FoxP3, which is required for imparting T cells with regulatory function. Furthermore, the loss of T-bet upon mTOR inhibition did not induce deviation into the type-2 or type-17 subset. These observations were also confirmed at varying doses of rapamycin (20 ng/ml-2 μg/ml). At higher doses, rapamycin efficiently blocked mTOR activity in OT-I cells (S6 Kp and S6p), but unlike CD4⁺T cells, it failed to block activation, proliferation, or deviation into regulatory T cell subsets. These results indicate that rapamycin has different effects on CD4⁺ and CD8⁺T cells, and its ability to block IL-12 induced type I CDS+effector differentiation is not because of induction of anergy or deviation to other effector subtypes.

### Example 7

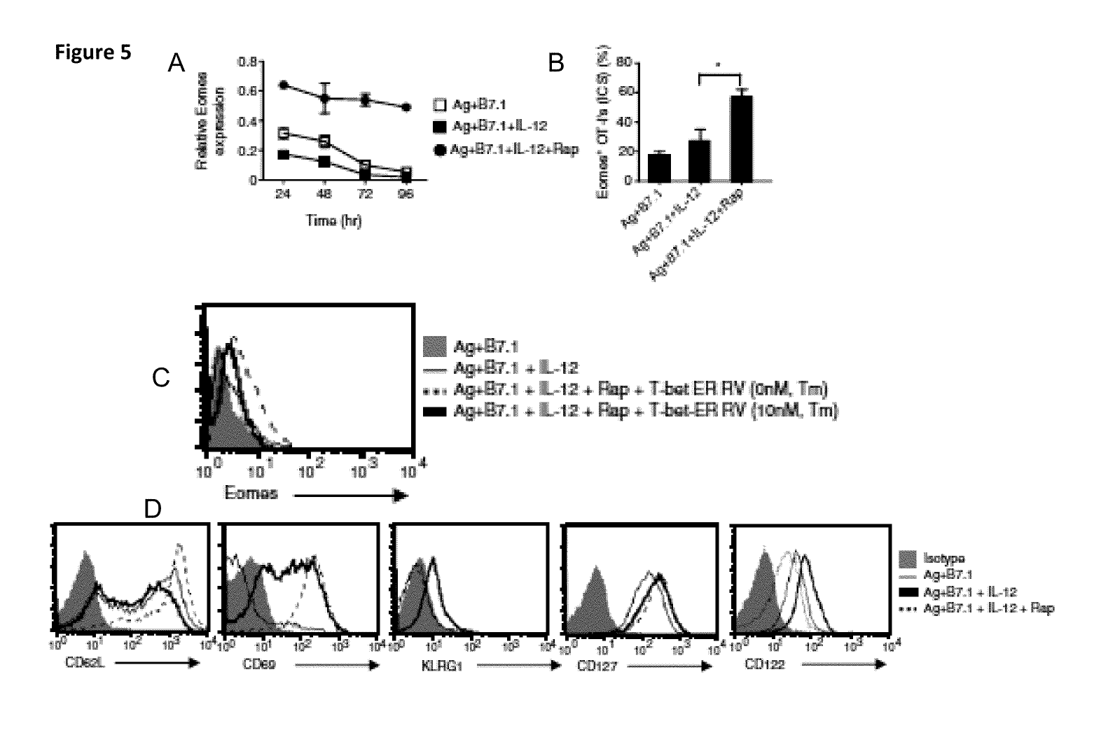

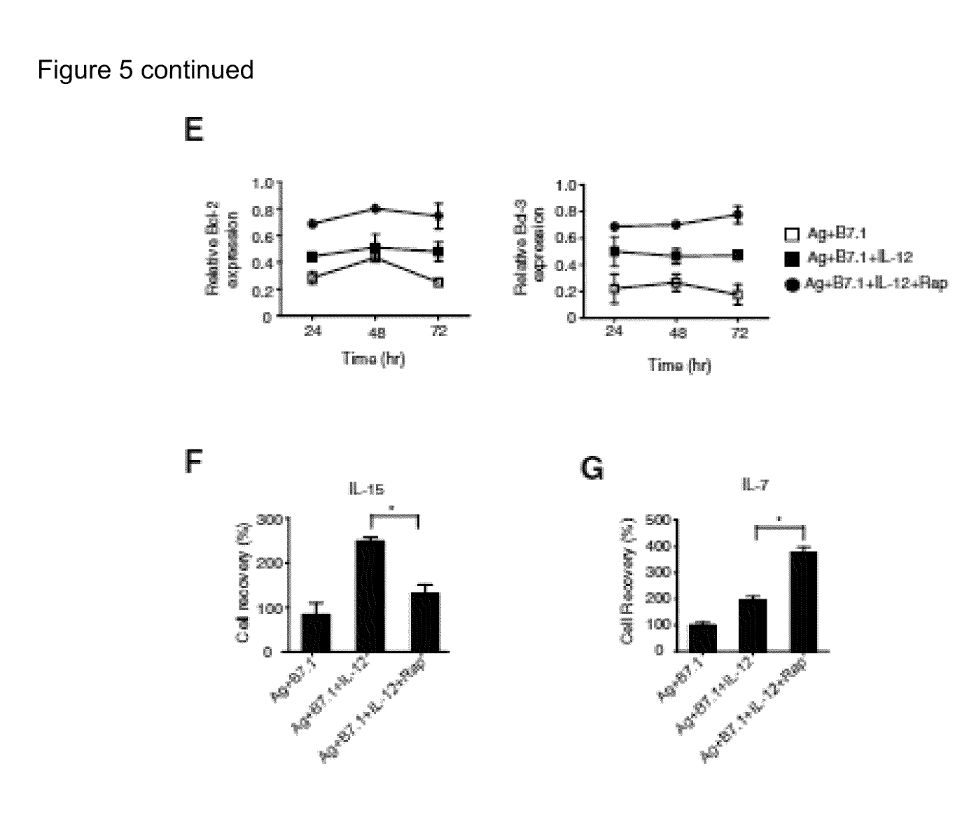

This Example demonstrates that mTOR inhibition induces persistent eomesodermin expression and produces memory-precursor CD8⁺T cells. Because rapamycin treatment blocked type I effector differentiation and failed to induce anergy or expression of other transcriptional regulators, we next sought to characterize the fate of rapamycin-treated IL-12-conditioned OT-I cells. The closely related transcription factors T-bet and Eomesodermin are inversely regulated in effector and memory CD8⁺T cells. To determine whether mTOR inhibition, which curtailed T-bet expression, led to induction of Eomesodermin, we systematically analyzed Eomesodermin mRNA expression in OT-I cells. We observed modest Eomesodermin expression in naive OT-I cells, which was enhanced when stimulated with Ag+B7.1 and reduced upon IL-12 addition (FIG. 5A). However, addition of rapamycin to Ag+B7.1 plus IL-12-conditioned OT-I cells markedly enhanced Eomesodermin mRNA expression, which was maintained at all time points tested (24-96 hr) (FIG. 5A). The increase in Eomesodermin mRNA was confirmed at the protein level because rapamycintreated IL-12-conditioned OT-I cells produced significant increases in Eomesodermin protein (FIG. 5B). It is noteworthy that we consistently observe marginal increases (nonsignificant) in Eomesodermin protein expression without mRNA induction in Ag+B7.1 plus IL-12-conditioned OT-I cells (FIGS. 5A and 5B). To test whether rapamycin-mediated upregulation of Eomesodermin in OT-I cells is a direct consequence of mTOR inhibition or a consequence of its ability to inhibit sustained T-bet expression, we ectopically induced T-bet expression in rapamycin conditioned OT-I cells and analyzed for Eomesodermin expression in the presence or absence of tamoxifen. Indeed, induction of T-bet in rapamycin-treated OT-I cells decreased Eomesodermin expression (FIG. 5C). Furthermore, we consistently observe increased Eomesodermin expression in Tbx21⁻¹⁻ OT-I cells treated with Ag+B7.1 and IL-12. Taken together, these results demonstrate that mTOR inhibition selectively switches the transcriptional program from sustained T-bet to Eomesodermin expression in IL-12-conditioned OT-I cells. We also determined whether IFN-α could also regulate mTOR activity and T-bet expression in OT-I cells. We determined that IFN-α was unable to enhance mTOR activity and T-bet expression in Ag+B7.1-stimulated OT-I cells; however, we observed increases in Eomesodermin expression and IFN-γ production. These results confirm that IL-12 has the unique ability to imprint type I effector maturation by promoting persistent mTOR and T-bet expression and that IFN-α may lack this activity because of its inability to promote persistent mTOR activity and mTOR dependent T-bet expression.

We next sought to determine whether rapamycin-induced switch in T-bet to Eomesodermin expression as well as a block in type I maturation resulted in their transition to memory precursors. We performed phenotypic analysis of OT-I cells using markers associated with memory precursor CD8+ T cells, i.e., CD62L (lymph node homing), CD69 (lymph node retention), CD 127 (IL-7R α; essential for memory T cell maintenance), CD 122 (IL-15β and essential for memory CD8+ T cell homeostatic renewal), KLRG1 (inversely corelated with memory CD8⁺ T cell generation), and Bcl-2 (antiapoptotic and increased expression in memory T cells). The IL-12-conditioned OT-I cells treated with rapamycin expressed markedly higher amounts of CD62L and also demonstrated persistent CD69 expression in comparison to non-rapamycin-conditioned cells (FIG. 5D). The increases in CD62L and CD69 expression imply that rapamycin-treated OT-I cells could have greater capacity for lymph node homing and retention. Moreover, rapamycin-treated cells had a higher frequency of KLRG1^{lo} cells compared to the non-treated controls, along with increased and sustained expression of prosurvival genes (Bcl-2 and Bcl-3) at all time points observed (FIGS. 5D and 5E). Thus, rapamycin treatment promotes a phenotype indicative of memory precursor CD8+ T cells. However, rapamycin treatment decreased CD122 expression, and the OT-I cells showed a defect in their ability to respond to IL-15 stimulation in vitro (FIGS. 5D and 5F). This is in agreement with the fact that rapamycin treatment causes a loss in T-bet expression and that CD122 is a direct gene target of T-bet in CD8+ T cells. Although we did not observe any changes in CD127 expression upon rapamycin treatment, these cells were better sensitized for IL-7 responsiveness in vitro (FIGS. 5D and 5G). Overall, these data indicate that mTOR inhibition imparts a memory-like phenotype on IL-12-conditioned effector CD8⁺ T cells along with persistent expression of memory fate transcription factor Eomesodermin.

We next investigated whether the reculture of 72 hr conditioned OT-I cells with IL-7 for an additional 72 hr or antigen recall (168 hr) affected their memory-like phenotype. We determined that rapamycin-treated OT-I cells maintained their CD62L^{hi} and KLRG1^{lo} phenotype, but the CD69^{hi} phenotype was lost. Notably, the CD122^{lo} phenotype observed at 72 hr was restored and we observed no changes in CD127 expression. Thus, resting the rapamycin-treated OT-I cells with IL-7 essentially maintained their memory-precursor phenotype, preventing their ability to maintain the CD69^{hi} phenotype.

### Example 8

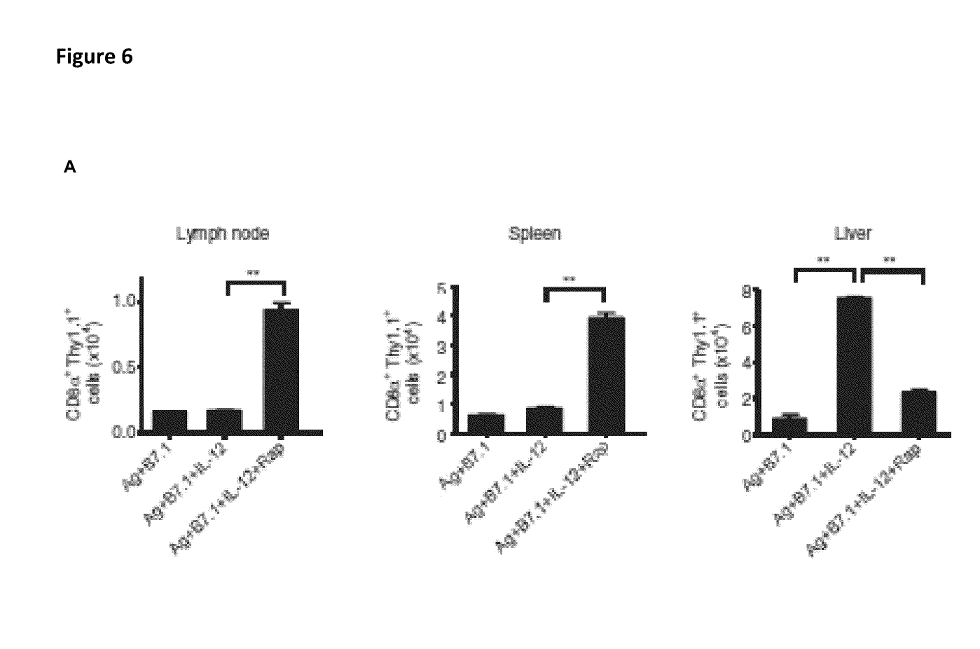

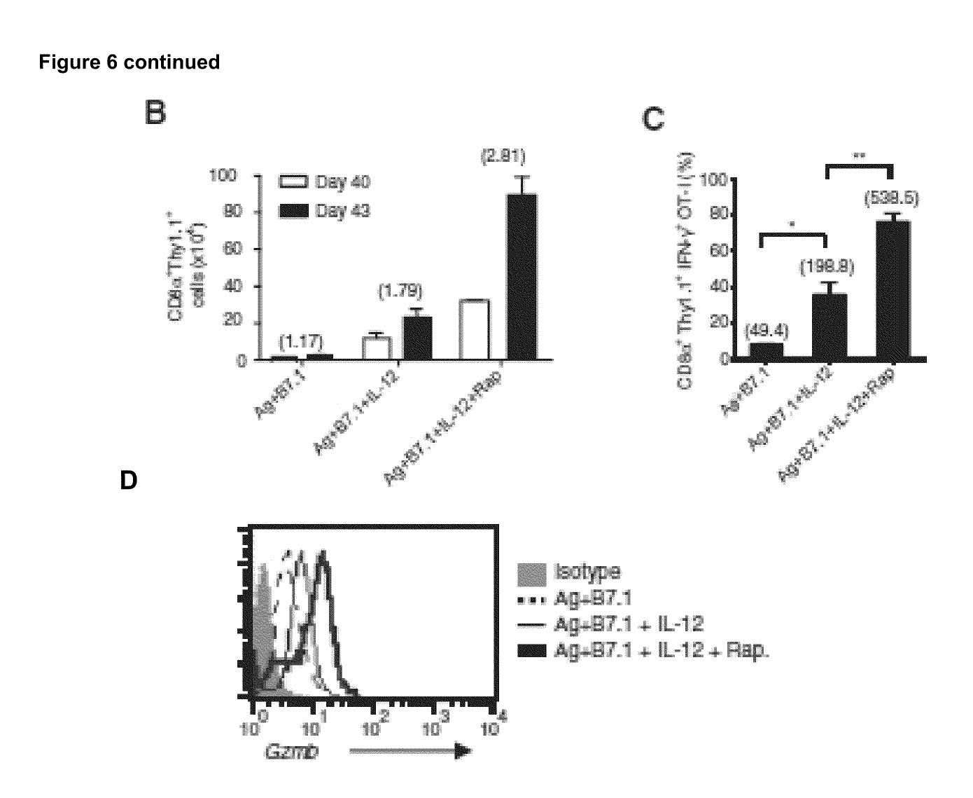

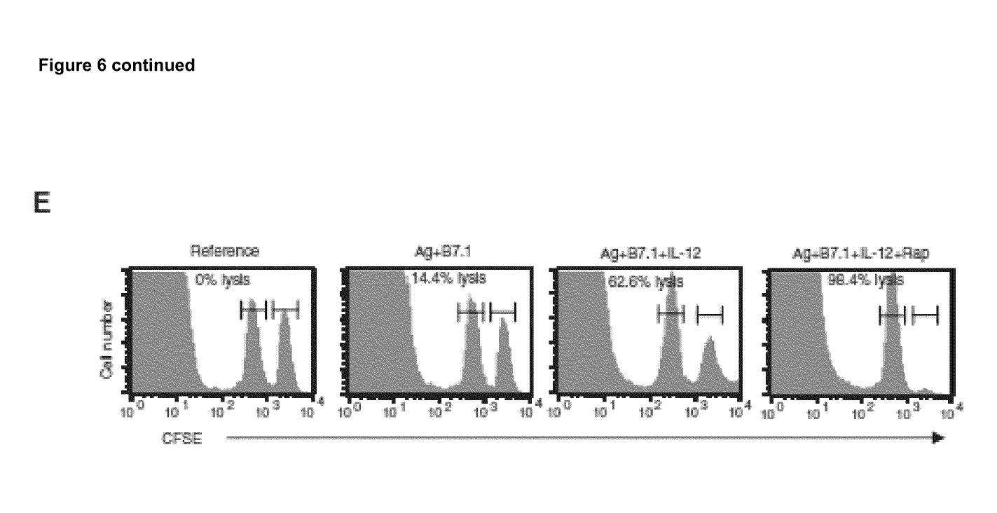

This Example demonstrates that inhibition of mTOR enhances memory CD8⁺ T cell generation. Based on the ability of rapamycin to block IL-12-mediated type I effector functions, switch persistent T-bet for Eomesodermin expression, and induce memory-like phenotype in OT-I cells, we analyzed whether rapamycin-treated IL-12-conditioned OT-I cells would produce memory responses after adoptive transfer. To test this, we first investigated if rapamycin treated OT-I cells show changes in their ability localize within secondary lymphoid organs as suggested by their increased CD62L and CD69 expression. The adoptively transferred Ag+B7.1±IL-12 and rapamycin-conditioned OT-I cells (Thy1.1⁺) were detected in C57BL/6 (Thy1.2⁺) recipients after 24 hr. The rapamycintreated OT-I cells demonstrated increased localization in secondary lymphoid compartments (lymph node and spleen) and correspondingly lesser numbers were observed in tertiary sites such as liver (FIG. 6A) and blood. The nonrapamycin-treated OT-I cells did not show this pattern of localization (FIG. 6A). However, we did not observe any significant differences in the frequency of cells in the lung. Thus, a block in mTOR activity shifts the localization of antigen plus IL-12-conditioned CD8⁺T cells to the secondary lymphoid compartment.

To confirm whether rapamycin treatment that produces memory precursor OT-I cells enables them for memory functions, we evaluated the persistence of the adoptively transferred cells (day 40) and tested their antigen recall response (day 43). The OT-I cells conditioned with Ag+B7.1 plus IL-12 demonstrate greater persistence than Ag+87.1-stimulated OT-I cells (FIG. 6B). However, rapamycin treatment markedly enhanced the ability of OT-I cells to persist, as demonstrated by the increased numbers detected on day 40 (FIG. 6B). The increased persistence of OT-I cells was largely because of their differential ability to survive rather than undergo greater homeostatic proliferation, as rapamycin-treated OT-I cells show identical CFSE dilution as the nontreated controls but have higher expression of survival-associated gene expressions (FIG. 5E). Moreover, the rapamycin-treated OT-I cells produced vigorous antigen recall responses as assessed by clonal expansion upon antigen rechallenge (FIG. 6B) and effector responses: IFN-γ, Granzyme 8 expression, and CTL activity (FIGS. 6C, 6D, and 6E). More importantly, there is increased expression of IFN-γ and Granzyme 8 on a per-cell basis in the rapamycin-treated group, which indicates that the increases in vivo cytolytic killing observed in this group is not only because of increased cell numbers, but also because of increased effector maturation upon antigen-recall. Therefore, rapamycin treatment not only enhances CD8⁺T cell persistence, but also empowers them for greater effector capacities upon antigenic rechallenge. Phenotypic analysis of the adoptively transferred OT-I cells at early (day 5) and late (day 40; memory) time points show that rapamycin-treated cells have higher CD127, CD62L, and CD69 expression on day 5, maintaining their memory precursor phenotype, but this phenotype was altered at day 40 posttransfer. In addition, no changes in T-bet and CD122 expression were noted on day 40. Collectively, these observations demonstrate that rapamycin treatment promotes CD8⁺T cell memory precursor generation that can localize within the secondary compartments and persist upon adoptive transfer. However, they alter their phenotype over time and produce robust antigen-recall effector responses.

### Example 9

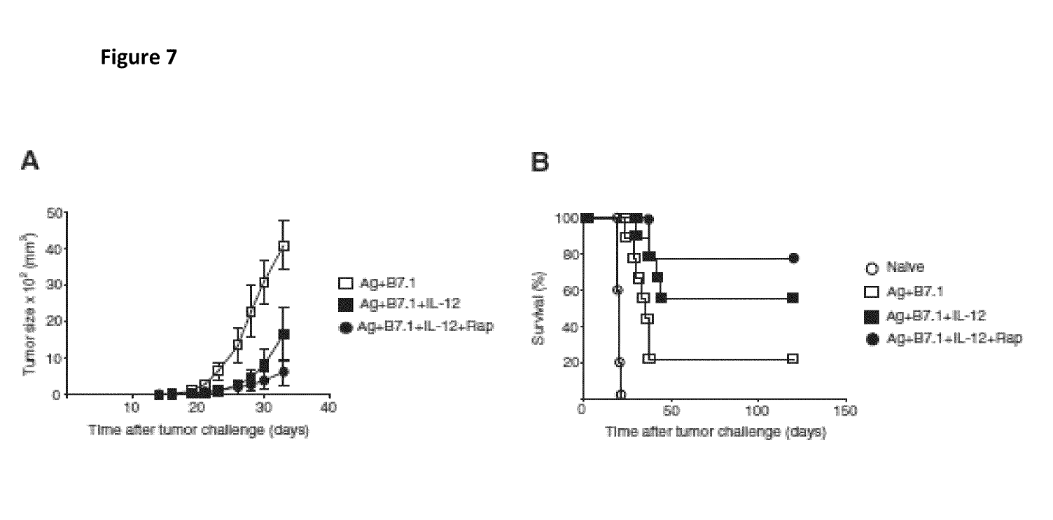

This Example shows that rapamycin-treated 11-12-conditioned OT-I cells have augmented tumor efficacy. The use of ex vivo generated tumor-antigen-specific effector CD8⁺ T cells in adoptive cell transfer (ACT) has produced tumor regressions in the clinical setting (Morgan et al., 2006). To test the tumor efficacy of rapamycin-treated IL-12-conditioned OT-I cells, we adoptively transferred IL-12-conditioned OT-I cells (72 hr) that were either treated with or without rapamycin into intact C57BL/6 recipients bearing E.G7 tumor cells and their tumor size (s.c.), and survival was monitored over time. In comparison to naive OT-I cell recipients, the mice receiving Ag+B7.1-stimulated OT-I cells showed marginal benefits (100% to 80% fatality by day 30), which was further enhanced by the IL-12-conditioned OT-I cells (50% fatality by day 30). Rapamycin-treated IL-12-conditioned OT-I cells showed markedly enhanced tumor efficacy as more than 78% of the recipient animals survived tumor-free till day 120 (FIG. 7B). Moreover, the rapamycin-treated IL-12-conditioned OT-I cells also show markedly enhanced control of tumor size when compared to non-rapamycin-treated counterparts (FIG. 7A). These results demonstrate that inhibition of mTOR programs antigen and IL-12-conditioned CD8⁺ T cells for memory responses that show greater tumor efficacy than IL-12-conditioned effector CD8⁺ T cells.

### Example 10

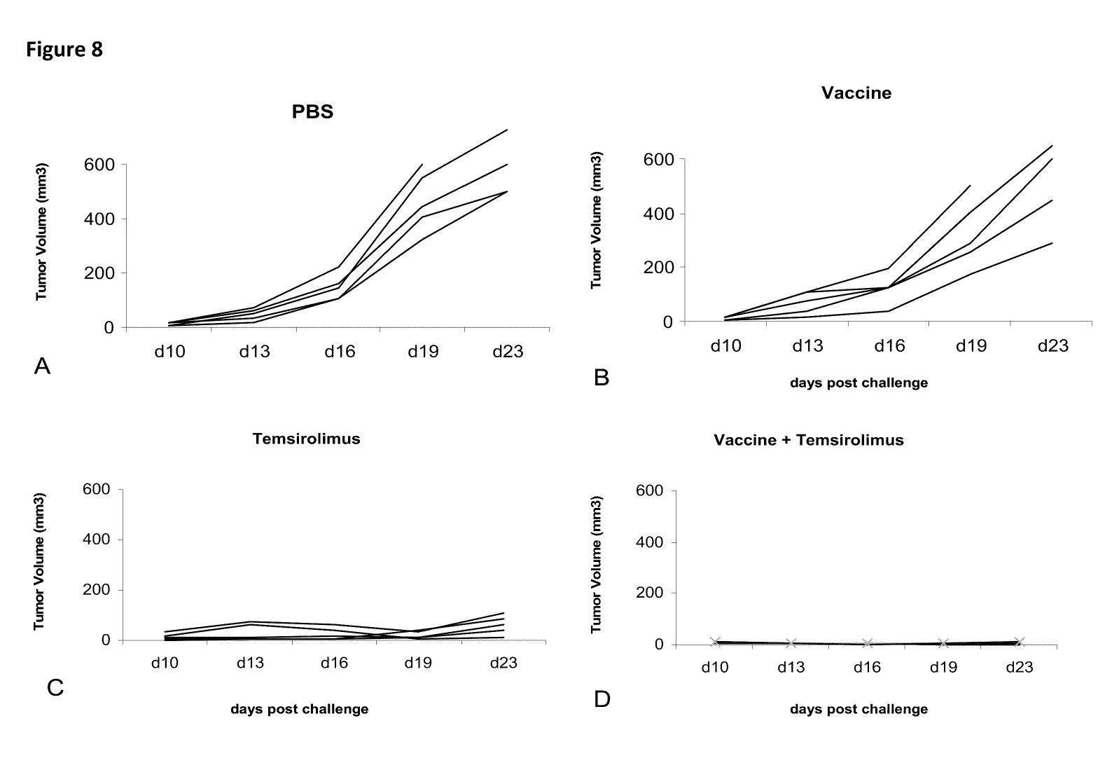

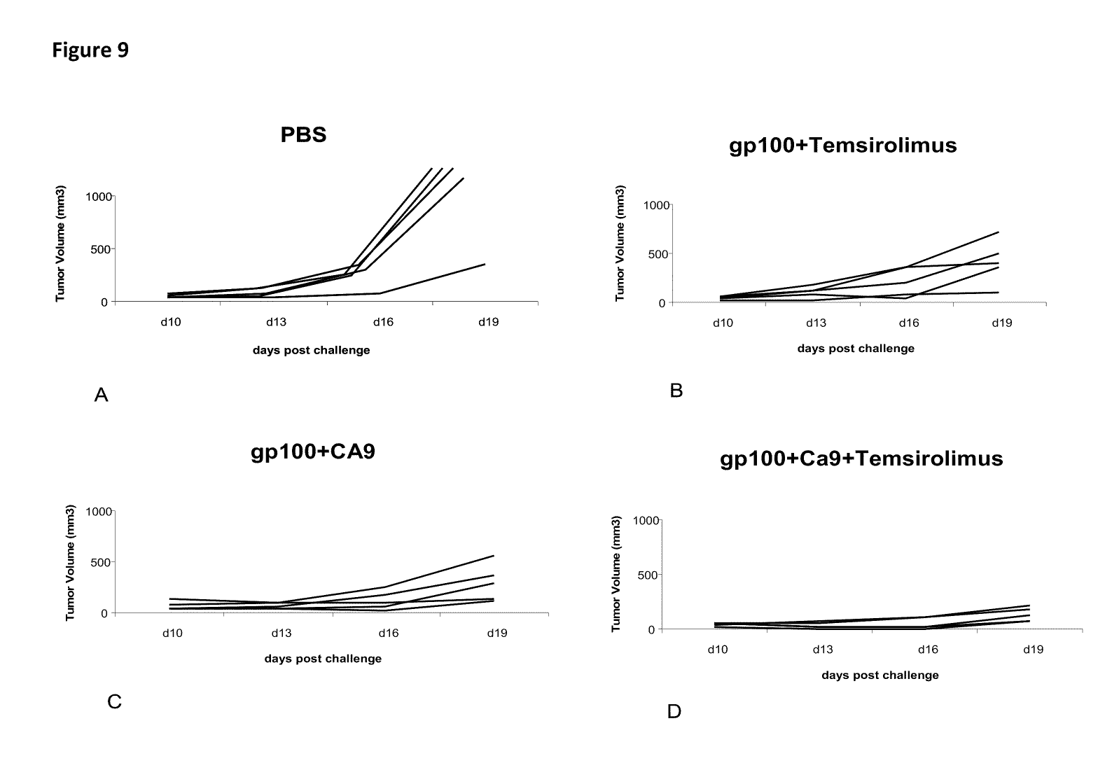

This Example demonstrates that temsirolimus and rapamycin enhance the antitumor effects of cancer vaccines in murine models for RCC and melanoma. In the RCC model, a heat shock protein (HSP) served as an immune adjuvant and was complexed to a target antigen, carbonic anhydrase IX (CA9), which is expressed by 90% of clear cell RCCs. Balb/c mice were implanted with syngeneic RENCA tumors engineered to express CA9. In a treatment model targeting established tumor implants, mice were treated 10 days after implantation with tumor vaccine with or without temsirolimus (FIG. 8). As can be seen from FIG. 8, the vaccine alone had only a modest effect on tumor growth. Temsirolimus alone produced a decrease in tumor growth, but the combination of vaccine and temsirolimus had the greatest effect on tumor growth. Similarly, in a melanoma model, the combination of vaccine and temsirolimus had the greatest effect on tumor growth (FIG. 9). In this model, CA9 was complexed to a melanoma antigen, gp100. C57/BL6 mice were implanted with syngeneic B16 tumors engineered to express gp100; mice were treated 10 days later with tumor vaccine. Similar results were obtained using a murine ovarian cancer model where the vaccine was augmented with rapamycin.

### Example 11

This Example demonstrates that the enhancement effect of temsirolimus is immune mediated. In particular, temsirolimus had a direct effect on the growth of RENCA (renal cancer cells) in vitro but had no effect on in vitro growth of B16 melanoma (FIG. 10). This indicated that in the melanoma model the primary effect of temsirolimus is immune mediated. Consistent with this possibility, immunization with CA9+gp100 elicited a gp100-specific IFN-γ response from splenocytes using an ELISPOT assay. This response was significantly augmented by concurrent treatment with temsirolimus (p<0.05). Further, specific killing increased with temsirolimus treatment in an in vivo CTL assay. Pmel-1 cells were adoptively transferred to C57/BL6 mice and immunized with gp100+CA9 with or without temsirolimus. Pmel-1 cells are transgenic cells that recognize the H-2 Db-restricted epitope corresponding to amino acids 25-33 of gp100.13 Target cells loaded with the H-2 Db-restricted epitope were injected and monitored 14 hours later by flow cytometry. Specific killing in the group that did not receive temsirolimus was 66%. When temsirolimus was administered with the vaccine, specific killing increased to 78%.

### Example 12

This Example illustrates various embodiments of the invention, each of which demonstrates the use of an mTOR inhibitor to enhance an anti-cancer immune response. In each case, Black 6 mice are used.

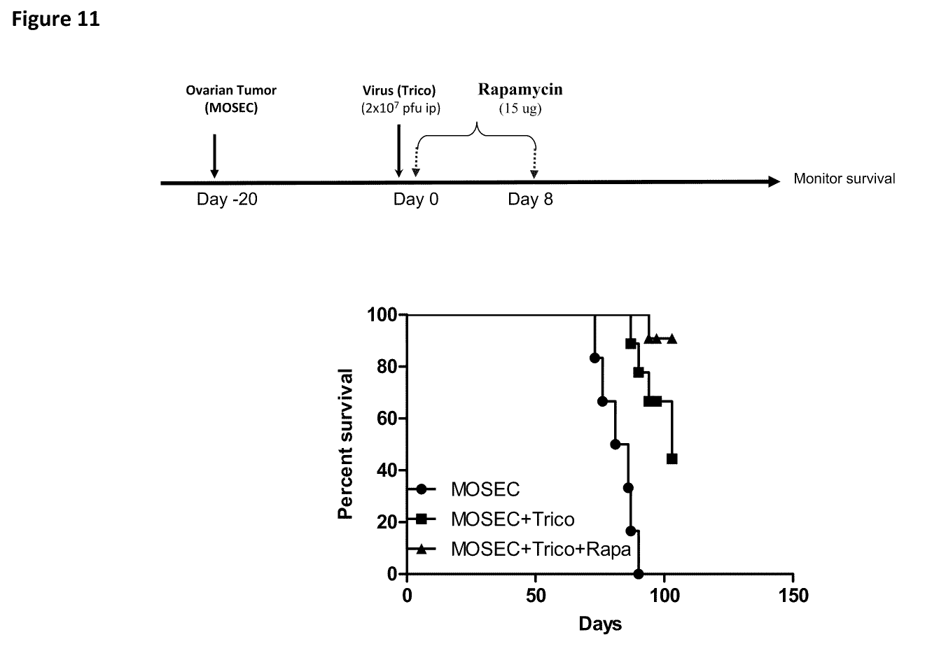

The data depicted in FIG. 11 demonstrate that rapamycin enhances immunization mediated protection against an established ovarian tumor. Briefly, the day 20 ovarian tumor bearing mice were created by injection of murine ovarian serous epithelial cells (“MOSEC”). The immunization was performed using a fowlpox based viral vector (“Trico” which is also referred to as “Tricom” (Sanofi Pasteur) expressing a chicken ovalbumin antigen in an MHC-I context. The virus also expresses three costimulatory molecules (B7.1, LFA3 and LFA-1) that participate in the activation of T cells (e.g., see Garnett, et al. Curr Pharm Des. 2006; 12(3):351-61, which is hereby incorporated by reference). The survival of the tumor bearing mice was monitored. Each experimental group had 20 mice and the experiment was repeated twice. As can be seen from the data in FIG. 11, the addition of rapamycin has a profound enhancing effect on immunization mediated survival of the tumor bearing mice, relative to the control groups.

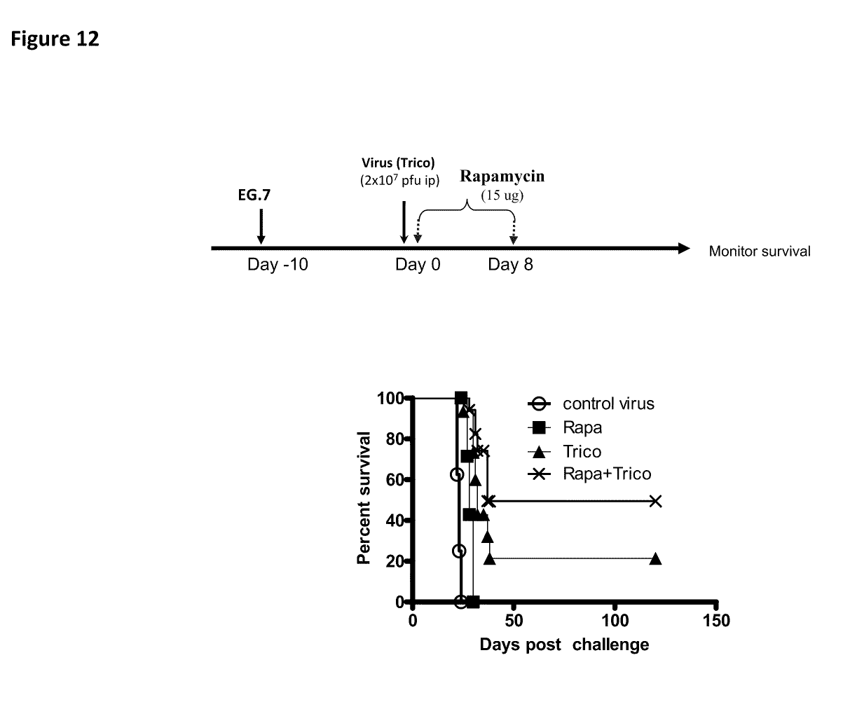

The data depicted in FIG. 12 demonstrate that mTOR inhibitor administration augments viral immunization mediated survival of thymoma bearing mice. The data summarized in FIG. 12 reflect analysis of mice that were inoculated with murine T cell thymoma chicken albumin expressing cells (EG.7) in the using the same experimental context as described for FIG. 11. It can be seen from these data that combining an mTOR inhibitor (rapamycin) with vaccination can significantly enhance survival of the tumor bearing mice.

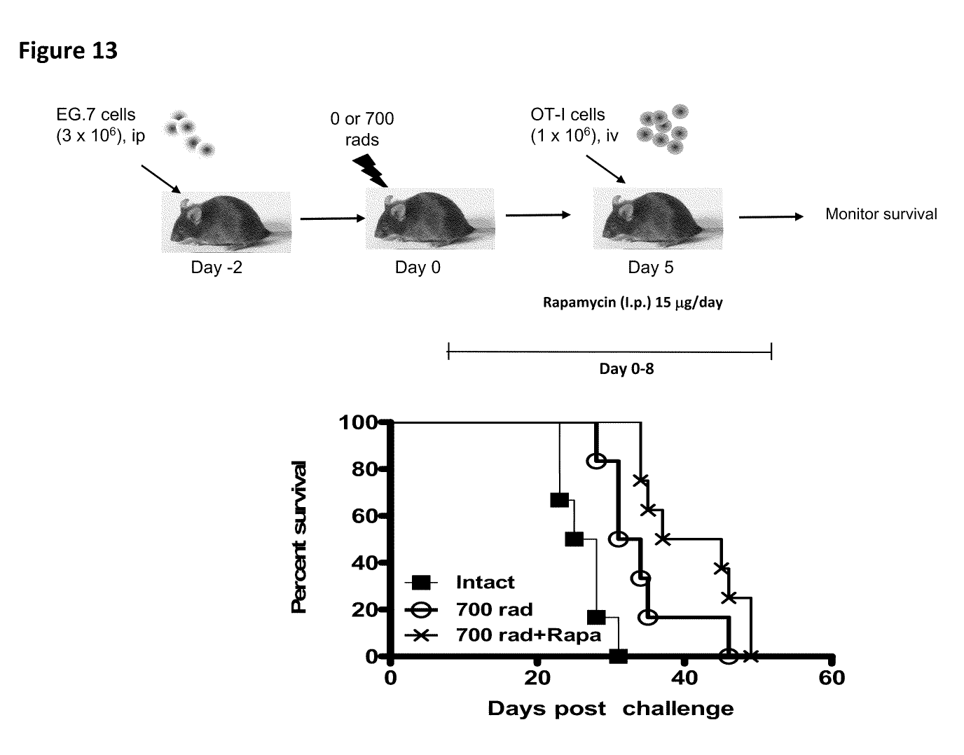

The data depicted in FIG. 13 illustrate that the addition of an mTOR inhibitor can enhance homeostatic proliferation (HP) induced anti-tumor immunity. In particular, radiation induced lymphopenia induces HP in naïve CD8+T cells, which produces functional maturation and memory. In tumor (thymoma-EG.7) bearing mice, radiation followed by adoptive transfer of naive tumor-antigen specific CD8+T cells generates protection against the growing tumor. As demonstrated in FIG. 13, this HP-induced tumor immunity is enhanced when rapamycin is administered such that the naïve CD8+T cells are matured by lymphopenia in the presence of rapamycin. Thus, the present invention is effective in enhancing the effects of a variety of induced immune responses against cells bearing cancer antigens.

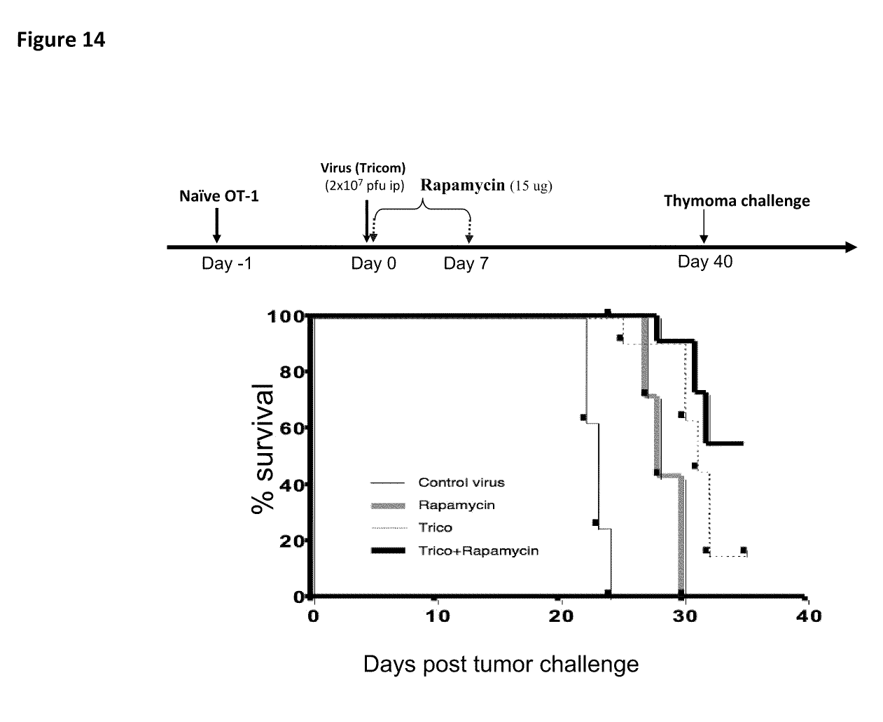

FIG. 14 provides a graphical summary of data demonstrating an enhanced prophylactic effect of the present invention. These data are generated in part using OT-1 cells. Briefly, OT-1 cells are obtained from the widely used transgenic OT-1 mouse in which all the CD8+T cells express a TCR specific for a peptide of ovalbumin presented on k^{b}. The amino acid sequence of the peptide is known in the art.

As shown in FIG. 14, naïve OT-1 cells are injected into naïve syngenic mice, after which the naïve recipient mice are immunized against the ovalbumin antigen using the Tricorn virus construct described above. Subsequent to the immunization, the mTOR inhibitor (rapamycin) is given daily for seven days. The graph shown in FIG. 14 has at its “0” the first day of thymoma challenge (day 40). Remarkably, the data indicate that the rapamycin treatment significantly enhances the survival f viral immunized mice when challenged by syngeneic tumor after 40 days. This represents the ability to generate memory CD8 T cells for durable tumor immunity and deterrence. Thus, the present invention provides a powerful method for prophylactic immunization, which could be employed, for example, in individuals at risk for developing cancer, as well as for those at risk for recurrence.

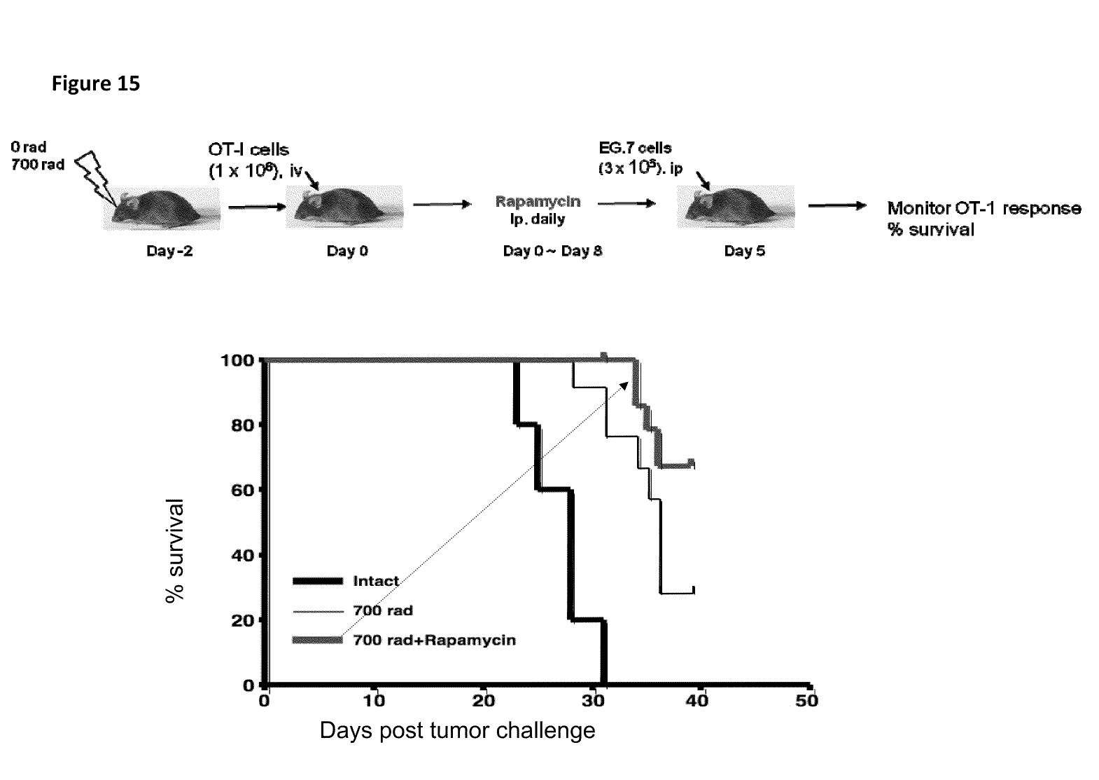

FIG. 15 provides data that demonstrate mTOR treatment enhances HP-induced anti-tumor CD8+T cell responses. In particular, as shown in FIG. 15, the C57BL/6 mice were irradiated and their CD8+T cell population reconstituted with OT-1 CD8+T cells. Rapamycin was administered daily for 8 days, after which the mice were challenged with EG.7 cells (thymoma cells expressing the albumin antigen). The use of the mTOR inhibitor again enhances the HP-induced tumor immunity as shown in a prophylactic immune response represented by the +rapamycin line.

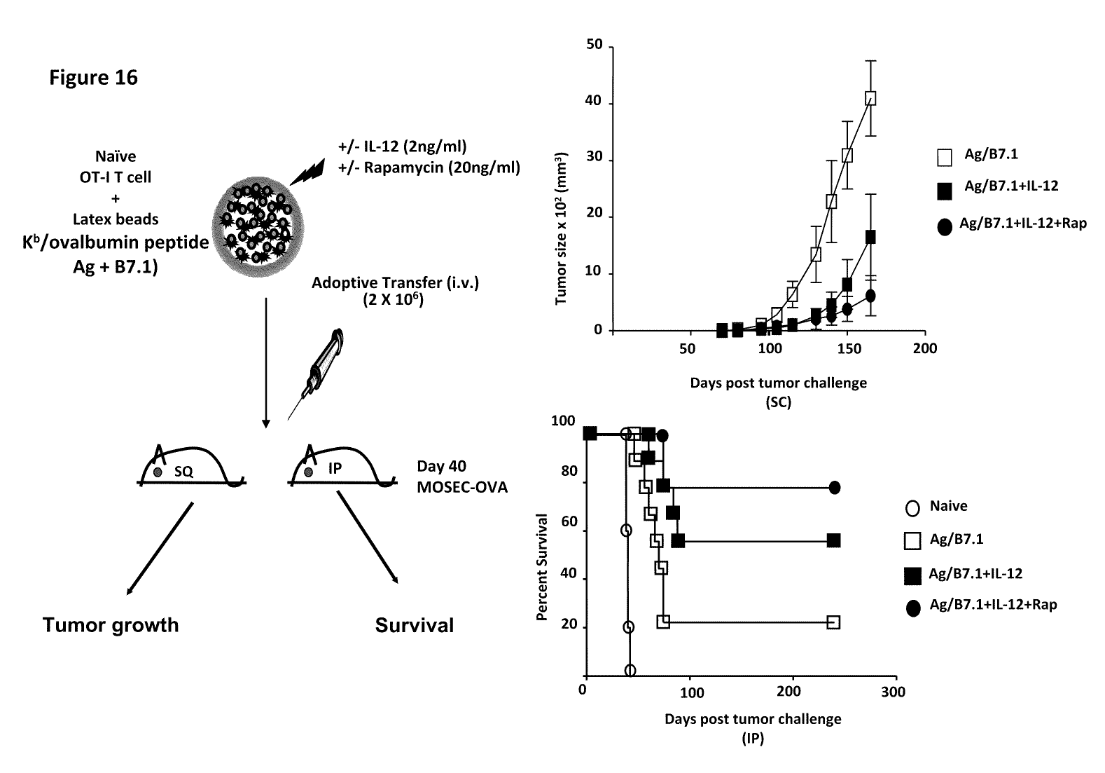

FIG. 16 demonstrates that the invention facilitated enhancement of CD8+T cell mediated ACT (Adoptive Cell Therapy) therapy of ovarian tumors. In particular, naïve OT-1 cells are incubated with the antigen in association with latex beads and the C57BL/6 murine equivalent of MHC Class I (H-210 in the presence or absence of IL-12 and an mTOR inhibitor (rapamycin) for 72 hours. The ex vivo generated antigen specific CD8+T cells are harvested and injected into syngeneic recipients bearing tumor (40 days), the adoptive transfer approach is used in mice created to have MOSEC-Ova tumors via either s.c. or i.p. routes. The s.c. injection yields tumors that are amenable to having their size measured, while the i.p. route yields data useful for determining survival time, which are summarized in the accompanying graphs. The data demonstrate a durable ability to control ovarian tumor challenge (at day 40) and promote survival. Thus rapamycin treated antigen plus co-stimulated fully activated CD8+T cells promote ovarian tumor immunity by adoptive cell transfer in a manner analogues to thymoma protection. The mice rendered tumor free up to day 300 show resistance to re-challenge thus indicative of memory T cells.

We claim:

1. A composition comprising an isolated population of CD8+T cells and an inhibitor of mammalian target of rapamycin (mTOR).

2. The composition of claim 1, wherein the CD8+T cells comprise at least 10% of the cells in the composition.

3. The composition of claim 1, further comprising an antigen to which the CD8+T cells are specific and to which an enhanced immune response in an individual is desired.

4. The composition of claim 3, further comprising interleukin-12 (IL-12).

5. The composition of claim 3, wherein the antigen is a tumor antigen.

6. A method for obtaining an enhanced immune response to an antigen in an individual in need of the enhanced immune response comprising administering to the individual the antigen and an inhibitor of mammalian target of rapamycin (mTOR).

7. The method of claim 6 wherein the individual has been diagnosed with or is suspected of having a cancer, wherein the cancer comprises cancer cells that express the antigen.

8. The method of claim 6, wherein the inhibitor of mTOR is administered to the individual subsequent to the administration of the antigen.

9. The method of claim 8, wherein the inhibitor of mTOR is administered to the individual at least once a day for at least 7 days.

10. The method of claim 8, wherein the inhibitor of mTOR is administered to the individual for not more than 20 consecutive days.

11. The method of 6, wherein the inhibitor of mTOR is rapamycin or temsirolimus.

12. The method of claim 6, further comprising administering to the individual a composition comprising isolated CD8+T cells specific for the antigen.

13. The method of claim 12, wherein the antigen has been presented to the CD8+T cells prior to the administering the CD8+T cells to the individual.

14. The method of claim 13, wherein the CD8+T cells have been contacted with an inhibitor of mTOR prior to the administering the CD8+T cells to the individual.

15. The method of claim 12, wherein the CD8+T cells are isolated from the individual prior to the administering the composition comprising the CD8+T cells to the individual.
